SEQUENCE LISTING (30 sequences)

SEQ ID NO: 1 - DNA, 20 bp, Artificial Sequence
  primer “DG012”
ttaattaaggtaatacggac

SEQ ID NO: 2 - DNA, 20 bp, Artificial Sequence
  primer “DG011”
gacccacgctcaccggctcc

SEQ ID NO: 3 - DNA, 25 bp, Artificial Sequence
  primer “DG009”
acatggcatgctgataataatatcg

SEQ ID NO: 4 - DNA, 45 bp, Artificial Sequence
  primer “DG010”
aatctggagccggtgagcgtgggtcccgcggtatcattgcagcac

SEQ ID NO: 5 - DNA, 50 bp, Artificial Sequence
  primer “DG007”
attatagaagaccccccccgagaccctcgaactcgcatacctctgataac

SEQ ID NO: 6 - DNA, 56 bp, Artificial Sequence
  primer “DG008”
cgatattattatcagcatgccatgtccacctgcaggctcccctacccggtagaatt

SEQ ID NO: 7 - DNA, 65 bp, Artificial Sequence
  primer “DG001”
ccagtaccggtccgtattaccttaattaatggccgattcgctagctttcccatgattccttcata

SEQ ID NO: 8 - DNA, 26 bp, Artificial Sequence
  primer “DG002”
actagtaaaaaagcaccgactcggtg

SEQ ID NO: 9 - DNA, 46 bp, Artificial Sequence
  primer “DG003”
caccgagtcggtgcttttttactagtctgcagactggctgtgtata

SEQ ID NO: 10 - DNA, 40 bp, Artificial Sequence
  primer “DG004”
gtctcggggggggtcttctataatgtgtggattttgagtt

SEQ ID NO: 11 - DNA, 45 bp, Artificial Sequence
  primer “DG013”
accgagtcggtgcttttttactagtggattttggtcatgggtggc

SEQ ID NO: 12 - DNA, 54 bp, Artificial Sequence
  primer “DG015”
cgagggtctcggggggggtcttctataatgtgtggattggcagctctggcccgt

SEQ ID NO: 13 - DNA, 43 bp, Artificial Sequence
  primer “DG021”
ttgtggaaaggacggctagcggattttggtggtcatgggtggc

SEQ ID NO: 14 - DNA, 17 bp, Artificial Sequence
  primer “M13-Fw”
gtaaaacgacggccagt

SEQ ID NO: 15 - DNA, 40 bp, Artificial Sequence
  primer “DG020”
ttgtggaaaggacggctagcctgcagactggctgtgtata

SEQ ID NO: 16 - DNA, 19 bp, Artificial Sequence
  primer “DG006”
ggcacactggccatatcgg

SEQ ID NO: 17 - DNA, 39 bp, Artificial Sequence
  primer “NM_ABC001Fw”
ccatacgagcacattacggggtgctggctgagggaacat

SEQ ID NO: 18 - DNA, 37 bp, Artificial Sequence
  primer “NM_ABC001Rv”
cttgactgagcgactgaggccccgctgctgaaaaacc

SEQ ID NO: 19 - DNA, 18 bp, Artificial Sequence
  primer “NM_ABC_gt_1_Fw”
gtgctggctgagggaaca

SEQ ID NO: 20 - DNA, 20 bp, Artificial Sequence
  primer “NM_ABC_gt_1_Rv”
ccccgctgctgaaaaaccca

SEQ ID NO: 21 - DNA, 95 bp, Artificial Sequence
  primer “BC_0074”
  misc_feature (62)..(64): n is a, c, g, or t
caagcagaagacggcatacgagatcggtctcggcattcctgctgaaccgctcttccgatctnnngtcgatgcataacttacggagtcgctctacg

SEQ ID NO: 22 - DNA, 92 bp, Artificial Sequence
  primer “BC_0075”
  misc_feature (59)..(61): n is a, c, g, or t
aatgatacggcgaccaccgagatctacactctttccctacacgacgctcttccgatctnnngtcgatgcaggatgggattctttaggtcctg

SEQ ID NO: 23 - DNA, 73 bp, Artificial Sequence
  primer “TALE_rptuinit1L”
agtaggagctcggtctcggtggcaatcgcgagtaacgggggcggcaaacaagcgtctgtcttcaccggttctg

SEQ ID NO: 24 - DNA, 73 bp, Artificial Sequence
  primer “TALE_rptuinit2L”
agtaggagctcggtctcggcgttagaaacggttcagcgtctcctgcccgtgctgtctgtcttcaccggttctg

SEQ ID NO: 25 - DNA, 76 bp, Artificial Sequence
  primer “TALE_rptuinit3L”
agtaggagctcggtctcgctgtgccaggcccacgggctgacgccgcagcaagtggtggctgtcttcaccggttctg

SEQ ID NO: 26 - DNA, 73 bp, Artificial Sequence
  primer “SpecR_CmR_common_RV ”
ccaagcttgcatgcctgcaggtcgacggtctcgtgacgtgtcttcgctagctaatgtgtggattttgagttag

SEQ ID NO: 27 - DNA, 56 bp, Artificial Sequence
  primer “ccdBCmR_Fw”
taggagctcggtctccctgagagtcttcaccggtctgcagactggctgtgtataag

SEQ ID NO: 28 - DNA, 73 bp, Artificial Sequence
  primer “TALE_rptuinit1R”
tcaagctagcgaagactcgtggcaatcgcgagtaacgggggcggcaaacaagcgtggagaccgtcgactgatc

SEQ ID NO: 29 - DNA, 73 bp, Artificial Sequence
  primer “TALE_rptuinit2R”
tcaagctagcgaagactcgcgttagaaacggttcagcgtctcctgcccgtgctgtggagaccgtcgactgatc

SEQ ID NO: 30 - DNA, 76 bp, Artificial Sequence
  primer “TALE_rptuinit3R”
tcaagctagcgaagactcctgtgccaggcccacgggctgacgccgcagcaagtggtggggagaccgtcgactgatc

(19) United States
(12) Patent Application Publication
(10) Pub. No.: US 2024/0043834 A1
(43) Pub. Date: Feb. 8, 2024

(54) LINKED DNA PRODUCTION METHOD AND VECTOR COMBINATION FOR USE THEREIN

(71) Applicant: THE UNIVERSITY OF TOKYO, Tokyo (JP)

(72) Inventors: Nozomu YACHIE, Bunkyo-ku, Tokyo (JP); Hideto MORI, Bunkyo-ku, Tokyo (JP); Nanami YAMAGUCHI, Bunkyo-ku, Tokyo (JP)

(73) Assignee: THE UNIVERSITY OF TOKYO, Tokyo (JP)

(21) Appl. No.: 17/913,723

(22) PCT Filed: Jan. 28, 2021
(86) PCT No.: PCT/JP2021/003060

§ 371 Date: Feb. 2, 2023

(30) Foreign Application Priority Data
Mar. 24, 2020 (JP) 2020-053077

(51) Int. Cl. C12N 15/11; C12N 15/64

(52) U.S. Cl. CPC C12N 15/11; C12N 15/64

(57) ABSTRACT

A method for producing a ligated DNA formed by ligating DNA fragments is disclosed. The method includes (a1) preparing (1) a first vector containing structure 5′-R1-D(i)-R2-M1-R2′-D(ii)-R1′-3′ and (2) a second vector containing structure 5′-R1-D(iii)-R2-M2-R2′-D(iv)-R1′-3′, which each contain recognition sequences of restriction enzymes R1, R1′, R2, and R2; selectable marker genes M1 and M2 different from each other; and DNA fragments for ligation D(i) to D(iv); (b1) treating the first vector with first restriction enzyme and second restriction enzyme to obtain a first vector fragment composed of structure 5′-D(i)-R2-M1-R2′-D(ii)-3; (c1) treating the second vector with third restriction enzyme and fourth restriction enzyme to obtain a second vector fragment with removed structure 5′-R2-M2-R2′-3′; and (d1) ligating the first vector fragment obtained in b1 and the second vector fragment obtained in cl by a ligation reaction to generate a third vector containing structure (3) 5′-R1-D(i)₁-R2-M1-R2′-D(ii)₁-R1′-3′.

### TECHNICAL FIELD

The present invention relates to a method for producing a ligated DNA and vector combinations for use therein.

### BACKGROUND ART

In recent years, there have been increasing demands for the synthesis of long-chain DNAs including whole-genome synthesis in all fields related to gene synthesis, such as medicine, industry, and biology. In general, a long-chain DNA is prepared by ligating chemically synthesized short-chain DNA groups of about 200 bp. However, this process is not perfect, and in the case of synthesizing a longer-chain DNA, ligation of many short-chain DNAs is required, making it difficult to obtain the target product. The DNA assembly techniques developed so far can be broadly classified into two.

<DNA Assembly Technique Using Type IIS Restriction Enzyme>

One is a method that uses a ligase to ligate short-chain DNAs treated with restriction enzymes, as typified by the Golden Gate method and the like (such as Engler C., Kandzia R., Marillonnet S., A one pot, one step, precision cloning method with high throughput capability. PLoS One. 2008; 3 (11): e3647. doi:10.1371/journal.pone.0003647 (NPL 1)). Other examples of such method include the BioBrick method (such as Knight T., Idempotent Vector Design for Standard Assembly of Biobricks. hdl: 1721.1/21168 (NPL 2)) and the OGAB method (such as Tsuge K. et al., Method of preparing an equimolar DNA mixture for one-step DNA assembly of over 50 fragments. Sci Rep. 2015 May 20; 5: 10655. doi:10.1038/srep10655. (NPL 3)). These methods have an advantage that by preparing a vector having short-chain DNAs to be ligated, the short-chain DNA groups are ligated at once by ligation reaction without amplification of DNA fragments by PCR or the like, and also have a characteristic that the experimental processing is simple and the processing time is short. In the Golden Gate method, a type IIS restriction enzyme capable of cleaving a site away from the recognition sequence is used to excise a DNA fragment from a plasmid vector. Therefore, when both ends of a DNA fragment to be ligated are cleaved by a type IIS restriction enzyme having a recognition sequence on the outside thereof, short-chain DNAs with protruding ends without recognition sequence can be excised from the vector. Therefore, by using the Golden Gate method, seamless assembly can be performed in which the synthesized target product does not contain unnecessary recognition sequences.

On the other hand, the length of protruding ends produced by standard type IIS restriction enzymes is 4 bp, so that the variety of protruding ends designable is limited. Therefore, the number of fragments that can be ligated at one time is limited to about 10 fragments. Moreover, depending on the target sequence, it may be difficult to design a specific protruding end. In particular, when assembling repeat sequences, it is difficult to ensure the specificity of protruding end sequences between DNA fragments to be ligated because the same sequence appears many times. In addition, the short-chain DNAs ligated by the Golden Gate method are designed and synthesized so that the protruding ends are dedicatedly specific only to the targeted sequences, and for this reason a short-chain DNA used for a certain assembly cannot always be used for another assembly and thus is low in reusability as a resource. Furthermore, as the number of DNA fragments to be ligated increases, the probability is higher of generating a non-targeted product due to non-specific ligation or the like, resulting in increased labor and time required for quality inspection by the PCR method or Sanger sequencing method.

<DNA Assembly Technique Using Recombinant Sequence>

The other is a method that ligates short-chain DNAs having common sequences of about several tens of bp at their ends, typified by the Gibson Assembly method and the like (such as Gibson D. G. et al., Enzymatic assembly of DNA molecules up to several hundred kilobases. Nat Methods. 2009 May; 6 (5): 343-5. (NPL 4)). Examples of such method include In Fusion Assembly (such as Zhu B. et al., In-fusion assembly: seamless engineering of multidomain fusion proteins, modular vectors, and mutations. Biotechniques. 2007 Sep.; 43(3): 354-9. (NPL 5)) and the overlap PCR method. Unlike the method using restriction enzymes, these can perform ligation by recombination reaction via common sequences at both ends without requiring a design that uses restriction enzymes for assembly. Therefore, restrictions on array design are extremely small.

On the other hand, the number of DNA fragments that can be efficiently ligated at one time is about 10 or less. Therefore, when synthesizing a long-chain DNA, it is necessary to repeat the assembly of several DNA fragments. In this case, it takes a lot of time and effort to perform a quality inspection using the PCR method or the Sanger sequencing method to check whether the target product is correctly synthesized for each assembly. In addition, synthesized short-strand DNAs and intermediate products generated in the assembly process can only be ligated to fragments having a common sequence next thereto, so that it is difficult to use them for assembly other than the intended purpose, and their reusability as a resource is very low. Furthermore, it is less suitable for the assembly of repeat sequences than methods using type IIS restriction enzymes. In assembling repeat sequences, even when an attempt is made to design a specific common sequence of several tens of bp that binds only adjacent DNA fragments, that sequence appears in other DNA fragment sequences, producing partial binding between all fragments. Therefore, it is impossible to synthesize repeat sequences by controlling the number of repetitions and the order.

### CITATION LIST

### Non Patent Literature

[NPL 1] Engler C., Kandzia R., Marillonnet S., A one pot, one step, precision cloning method with high throughput capability. PLoS One. 2008; 3 (11): e3647. doi:10.1371/journal.pone.0003647
[NPL 2] Knight T., Idempotent Vector Design for Standard Assembly of Biobricks. hdl: 1721.1/21168
[NPL 3] Tsuge K. et al., Method of preparing an equimolar DNA mixture for one-step DNA assembly of over 50 fragments. Sci Rep. 2015 May 20; 5: 10655. doi:10.1038/srep10655.
[NPL 4] Gibson D. G. et al., Enzymatic assembly of DNA molecules up to several hundred kilobases. Nat Methods. 2009 May; 6 (5): 343-5.
[NPL 5] Zhu B. et al., In-fusion assembly: seamless engineering of multidomain fusion proteins, modular vectors, and mutations. Biotechniques. 2007 Sep.; 43 (3): 354-9.

### SUMMARY OF INVENTION

### Technical Problem

The present invention has been made in view of the above-mentioned problems of the prior art, and an object thereof is to provide a method for producing a ligated DNA capable of accurately and efficiently ligating several tens or more of DNA fragments easily, and vector combinations for use therein.

### Solution to Problem

The present inventors made earnest studies to achieve the above object and have found as a result that if two vectors (toolkit vectors) containing specific constructs with different selectable marker genes are used to incorporate the switching of these two different selectable markers into the sequential DNA fragment ligation process, several tens or more of DNA fragments can be ligated and accumulated accurately and efficiently in a short time easily, even for fragments in which the same sequence such as a repeat sequence appears many times. Thus, the present invention has been completed. That is, the present invention includes the following aspects.

[1]

A method for producing a ligated DNA formed by ligating DNA fragments, comprising:
(a1) a step a1 of preparing a first vector containing the following structure (1) and a second vector containing the following structure (2):

5′-R1-D(i)-R2-M1-R2′-D(ii)-R1′-3′ (1)

5′-R1-D(iii)-R2-M2-R2′-D(iv)-R1′-3′ (2)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one. The first restriction enzyme cleaves inside of R1 or a 3′-side of R1, and the second restriction enzyme cleaves inside of R1′ or a of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.];
(b1) a step b1 of treating the first vector with the first restriction enzyme and the second restriction enzyme to obtain a first vector fragment composed of the structure: 5′-D(i)-R2-M1-R2′-D(ii)-3′;
(c1) a step c1 of treating the second vector with the third restriction enzyme and the fourth restriction enzyme to obtain a second vector fragment with the removed structure: 5′-R2-M2-R2′-3′; and
(d1) a step d1 of ligating the first vector fragment obtained in step b1 and the second vector fragment obtained in step c1 by a ligation reaction to generate a third vector containing the following structure (3):

5′-R1-D(i)₁-R2-M1-R2′-D(ii)₁-R1′-3′ (3)

[Here, D(i)₁ represents a DNA fragment containing the following structure: 5′-D(iii)-D(i)-3′, and D(ii)₁ represents a DNA fragment containing the following structure: 5′-D(ii)-D(iv)-3′.].
[2]

The method for producing a ligated DNA according to [1], further comprising: after step d1, a step of transforming a ligation reaction product into a host; and a step of using expression of the first selectable marker gene as an index to select a host introduced with the third vector.

[3]

The method for producing a ligated DNA according to [1] or [2], further comprising: after step d1, a step of treating the third vector with the third restriction enzyme and the fourth restriction enzyme to remove the structure: 5′-R2-M1-R2′-3′, thereby generating a fifth vector containing the structure: 5′-R1-D(i)₁-D(ii)₁-R1′-3′.

[4]

The method for producing a ligated DNA according to any one of [1] to [3], further comprising: using the third vector generated in step d1 as the first vector in step a1 and repeating steps a1 to d1 for an additional n cycles (1+n cycles in total) to generate a third′ vector containing the structure (3′):

5′-R1-D(i)₁₊ₙ-R2-M1-R2′-D(ii)₁₊ₙ-R1′-3′ (3′)

[Here, D(i)₁₊ₙ represents a DNA fragment containing the structure obtained at cycle 1+n: 5′-D(iii)-D(i)ₙ-3′; D(ii)₁₊ₙ represents a DNA fragment containing the structure obtained at cycle 1+n: 5′-D(ii)ₙ-D(iv)-3′; n represents a natural number; between the cycles, D(iii) of the second vector may be the same or different from each other; and between the cycles, D(iv) of the second vector may be the same or different from each other.].
[5]

A method for producing a ligated DNA formed by ligating DNA fragments, comprising:
(a2) a step a2 of preparing a first vector containing the following structure (1) and a second vector containing the following structure (2):

5′-R1-D(i)-R2-M1-R2′-D(ii)-R1′-3′ (1)

5′-R1-D(iii)-R2-M2-R2′-D(iv)-R1′-3′ (2)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one. The first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.];
(b2) a step b2 of treating the second vector with the first restriction enzyme and the second restriction enzyme to obtain a second vector fragment composed of the structure: 5′-D(iii)-R2-M2-R2′-D(iv)-3′;
(c2) a step c2 of treating the first vector with the third restriction enzyme and the fourth restriction enzyme to obtain a first vector fragment with the removed structure: 5′-R2-M1-R2′-3′; and
(d2) a step d2 of ligating the second vector fragment obtained in step b2 and the first vector fragment obtained in step c2 by a ligation reaction to generate a fourth vector containing the following structure (4):

5′-R1-D(iii)₁-R2-M2-R2′-D(iv)₁-R1′-3′ (4)

[Here, D(iii)₁ represents a DNA fragment containing the following structure: 5′-D(i)-D(iii)-3′, and D(iv)₁ represents a DNA fragment containing the following structure: 5′-D(iv)-D(ii)-3′.].
[6]

The method for producing a ligated DNA according to [5], further comprising: after step d2, a step of transforming a ligation reaction product into a host; and a step of using expression of the second selectable marker gene as an index to select a host introduced with the fourth vector.

[7]

The method for producing a ligated DNA according to [5] or [6], further comprising: after step d2, a step of treating the fourth vector with the third restriction enzyme and the fourth restriction enzyme to remove the structure: 5′-R2-M2-R2′-3′, thereby generating a sixth vector containing the structure: 5′-R1-D(iii)₁-D(iv)₁-R1′-3′.

[8]

The method for producing a ligated DNA according to any one of [5] to [7], further comprising: using the fourth vector generated in step d2 as the second vector in step a2 and repeating steps a2 to d2 for an additional n cycles (1+n cycles in total) to generate a fourth′ vector containing the structure (4′):

5′-R1-D(iii)₁₊ₙ-R2-M2-R2′-D(iv)₁₊ₙ-R1′-3′ (4′)

[Here, D(iii)₁₊ₙ represents a DNA fragment containing the structure obtained at cycle 1+n: 5′-D(i)-D(iii)ₙ-3′; D(iv)₁₊ₙ represents a DNA fragment containing the structure obtained at cycle 1+n: 5′-D(iv)ₙ-D(ii)-3′; n represents a natural number; between the cycles, D(i) of the first vector may be the same or different from each other; and between the cycles, D(ii) of the first vector may be the same or different from each other.].

[9]

The method for producing a ligated DNA according to any one of [1] to [4], wherein the fourth vector generated in step d2 of [5] or the fourth′ vector generated in [8] is used as the second vector in step a1.

The method for producing a ligated DNA according to any one of [5] to [8], wherein the third vector generated in step d1 of [1] or the third′ vector generated in [4] is used as the first vector in step a2.

The method for producing a ligated DNA according to any one of [1] to [10], wherein
the first restriction enzyme is a type IIS restriction enzyme that cleaves the 3′-side of R1, and the second restriction enzyme is a type IIS restriction enzyme that cleaves the 5′-side of R1′, and/or
the third restriction enzyme is a type IIS restriction enzyme that cleaves the 5′-side of R2, and the fourth restriction enzyme is a type IIS restriction enzyme that cleaves the 3′-side of R2′.

The method for producing a ligated DNA according to any one of [1] to [11], wherein
a third selectable marker gene, which is a selectable marker gene with an opposite action to that of the first selectable marker gene, is further inserted between R2 and R2′ of the first vector, and/or
a fourth selectable marker gene, which is a selectable marker gene with an opposite action to that of the second selectable marker gene and can be the same as or different from the third selectable marker gene, is further inserted between R2 and R2′ of the second vector.

The method for producing a ligated DNA according to any one of [1] to [12], wherein
a recognition sequence of a fifth restriction enzyme different from R1, R1′, R2, and R2′ is further set at a site other than the structure (1) in the first vector, and
a recognition sequence of a sixth restriction enzyme different from R1, R1′, R2, R2′, and the recognition sequence of the restriction enzyme is further set at a site other than the structure (2) in the second vector.
   [14]

A vector combination for use in the method for producing a ligated DNA according to any one of [1] to [13], comprising:
a first vector containing the following structure (1) and a second vector containing the following structure (2):

5′-R1-D(i)-R2-M1-R2′-D(ii)-R1′-3′ (1)

5′-R1-D(iii)-R2-M2-R2′-D(iv)-R1′-3′ (2)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one. The first restriction enzyme cleaves inside of R1 or a 3′-side of R1, and the second restriction enzyme cleaves inside of R1′ or a 5′-slide of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.].

A vector combination for use in the method for producing a ligated DNA according to any one of [1] to [13], comprising:
a first vector containing the following structure (1′) and a second vector containing the following structure (2′):

5′-R1-E1-R2-M1-R2′-E2-R1′-3′ (1′)

5′-R1-E3-R2-M2-R2′-E4-R1′-3′ (2′)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; E1, E2, E3, and E4 each independently represent a DNA fragment insertion site; E1 and E2 may be either one, and E3 and E4 may be either one. The first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.].

### Advantageous Effects of Invention

According to the present invention, it is possible to provide a method for producing a ligated DNA capable of accurately and efficiently ligating several tens or more of DNA fragments easily, and vector combinations for use therein.

In addition, according to the present invention, even fragments in which the same sequence such as a repeat sequence appears many times can be continuously ligated and can be used as they are for another assembly, so that reusability is high as well. Furthermore, since the probability is low of generating a non-targeted product due to non-specific ligation or the like, it is also possible to reduce the labor and time required for quality inspection. According to the present invention, it is also possible to prepare a vector library for efficient multiple genome editing and a pooled library for isolating a desired clone by the PCR method.

### BRIEF DESCRIPTION OF DRAWINGS

FIG. 1 is a schematic diagram showing a mode of the ligation between D(iii) (3) and D(i) (1), and the ligation between D(iv) (4) and D(ii) (2).

FIG. 2 is a schematic diagram showing a mode of the ligation between D(iii) (3) and D(ii) (2).

FIG. 3 is a schematic diagram showing a mode of selecting a target product by a selectable marker gene and a restriction enzyme.

FIG. 4 is a schematic diagram showing an aspect of the first method of the present invention.

FIG. 5 is a schematic diagram showing an aspect of the second method of the present invention.

FIG. 6 is a schematic diagram showing an aspect of combination of the first method and the second method of the present invention.

FIG. 7 is a schematic diagram showing an aspect of reuse of the target product and the intermediate product obtained in each cycle of the first method and the second method of the present invention.

FIG. 8 is a schematic diagram showing an aspect of preparing a TALE repeat unit array.

FIG. 9 is a schematic diagram showing an aspect of preparing a library pool of TALE repeat unit arrays.

FIG. 10 is a schematic diagram showing toolkit vector 1 (n¹) obtained in the preparation of gRNA-BC vector.

FIG. 11 is a schematic diagram showing toolkit vector 2 (n²) obtained in the preparation of gRNA-BC vector.

FIG. 12 is a schematic diagram showing the gRNA-BC unit in the toolkit vector obtained at each step of preparing the gRNA-BC vector.

FIG. 13 is an electropherogram of fragments of the toolkit vector obtained at each step of preparing the gRNA-BC vector.

FIG. 14 is an electropherogram of vector fragments isolated from clones 1 to 6 obtained by transforming the ligation products in the preparation of the gRNA-BC vector.

FIG. 15 is a graph showing the editing efficiency of the top 26 sites with the highest base editing rates after transfection of Array vector lib, Single vector lib, and Single liner DNA lib.

### DESCRIPTION OF EMBODIMENTS

Hereinafter, the present invention is described in detail with reference to preferred embodiments thereof.

The present invention first provides the following first method and second method as methods for producing a ligated DNA formed by ligating DNA fragments.

### <First Method for Producing Ligated DNA>

The first method for producing a ligated DNA of the present invention includes:
(a1) a step a1 of preparing a first vector containing the following structure (1) and a second vector containing the following structure (2):

5′-R1-D(i)-R2-M1-R2′-D(ii)-R1′-3′ (1)

5′-R1-D(iii)-R2-M2-R2′-D(iv)-R1′-3′ (2)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one. The first restriction enzyme cleaves inside of R1 or a 3′-side of R1, and the second restriction enzyme cleaves inside of R1′ or a 5′-slide of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.];
(b1) a step b1 of treating the first vector with the first restriction enzyme and the second restriction enzyme to obtain a first vector fragment composed of the structure: 5′-D(i)-R2-M1-R2′-D(ii)-3′;
(c1) a step c1 of treating the second vector with the third restriction enzyme and the fourth restriction enzyme to obtain a second vector fragment with the removed structure: 5′-R2-M2-R2′-3′; and
(d1) a step d1 of ligating the first vector fragment obtained in step b1 and the second vector fragment obtained in step c1 by a ligation reaction to generate a third vector containing the following structure (3):

5′-R1-D(i)₁-R2-M1-R2′-D(ii)₁-R1′-3′ (3)

[Here, D(i)i represents a DNA fragment containing the following structure: 5′-D(iii)-D(i)-3′, and D(ii)₁ represents a DNA fragment containing the following structure: 5′-D(ii)-D(iv)-3′.].

(Step a1)

The first method of the present invention first prepares (step a1) a first vector containing the following structure (1):

5′-R1-D(i)-R2-M1-R2′-D(ii)-R1′-3′ (1)

and a second vector containing the following structure (2):

5′-R1-D(iii)-R2-M2-R2′-D(iv)-R1′-3′ (2)

-DNA Fragment For Ligation-

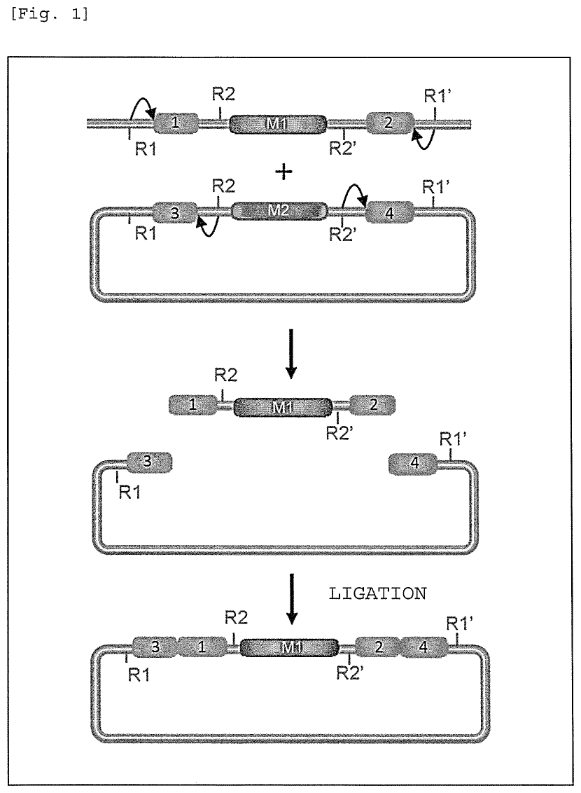

In the present invention, D(i) to D(iv) each independently represent a DNA fragment for ligation. In the first method of the present invention, D(i) is ligated to the 3′-side of D(iii) and D(ii) is ligated to the 5′-side of D(iv) through steps b1 to d1 described later. As a result, finally, on the 5′-side of the first selectable marker gene, D(iii) and D(i) are ligated, and on the 3′-side, D(iv) and D(ii) are ligated (FIG. 1).

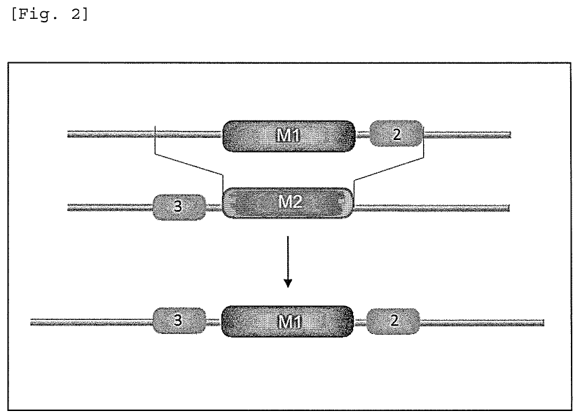

In the method of the present invention, D(i) and D(ii) may be either one, and D(iii) and D(iv) may be either one. In this aspect, for example, when D(i) and D(iv) are not present, D(iii) is eventually placed on the 5′-side of the first selectable marker gene and D(ii) is placed on the 3′-side (FIG. 2). In this case, D(iii) and D(ii) can finally be ligated by treatment with a third restriction enzyme and a fourth restriction enzyme.

Such D(i) to D(iv) are not limited as long as they do not contain the recognition sequence of the restriction enzyme or the selectable marker gene according to the present invention, and may be any DNAs, which may be the same or different from each other, and may have regularity such as containing sequences common to each other. The sizes of D(i) to D(iv) are also not particularly limited, and several bp to several tens of kbp can be ligated.

-Restriction Enzyme and Recognition Sequence Thereof-

In the present invention, R1 represents the recognition sequence of the first restriction enzyme, R1′ represents the recognition sequence of the second restriction enzyme, R2 represents the recognition sequence of the third restriction enzyme, and R2′ represents the recognition sequence of the fourth restriction enzyme. The first restriction enzyme and the second restriction enzyme may be the same or different, and the third restriction enzyme and the fourth restriction enzyme may be the same or different, but when comparing the first restriction enzyme and the second restriction enzyme with the third restriction enzyme, and the first restriction enzyme and the second restriction enzyme with the fourth restriction enzyme, they must be different restriction enzymes with different recognition sequences.

In step b1 described later, a DNA fragment having the structure “5′-D(i)-R2-M1-R2′-D(ii)-3′” is excised from the first vector, but in this step, when the first restriction enzyme or the second restriction enzyme recognizes R2 or R2′, D(i) and D(ii) are excised, making it impossible to obtain the target DNA fragment. In addition, in step c1 described later, the structure “5′-R2-M2-R2′-3′” in the second vector is removed by treatment with the third restriction enzyme and the fourth restriction enzyme, but when these restriction enzymes also recognize R1 or R1′ left in the second vector, D(iii) and D(iv) are excised from the second vector, and the ligation DNA (DNA for ligation) disappears from the second vector. Therefore, from the viewpoint of avoiding such inappropriate cleavage, the first restriction enzyme and the second restriction enzyme need to be different restriction enzymes from the third restriction enzyme and the fourth restriction enzyme (that is, the recognition sequences R1 and R1′ need to be recognition sequences different from R2 and R2′).

On the other hand, when the first restriction enzyme and the second restriction enzyme are the same (that is, when the recognition sequences R1 and R1′ are the same), treatment with a single restriction enzyme is preferable from the viewpoint that the target DNA fragment can be obtained in step b1 described later, and the operation is simple. Also, when the third restriction enzyme and the fourth restriction enzyme are the same as well (that is, when R2 and R2′ are the same), treatment with a single restriction enzyme is preferable from the viewpoint that it can remove the target structure in step c1 described later, and the operation is simple.

In the first method of the present invention, the first restriction enzyme cleaves inside of R1 or the 3′-side of R1, and the second restriction enzyme cleaves inside of R1′ or the 5′-side of R1′, without cleaving any other sites inside the first vector and the second vector (as well as the third, third′, fourth, and fourth′ vectors described later). In addition, the second restriction enzyme cleaves inside of R1′ or the 5′-slide of R1′, the third restriction enzyme cleaves inside of R2 or the 5′-side of R2, and the fourth restriction enzyme cleaves inside of R2′ or the 3′-side of R2′, each without cleaving any other sites inside the first vector and the second vector (as well as the third, third′, fourth, and fourth′ vectors described later).

The restriction enzyme cleaves inside the recognition sequence (inside of R1, inside of R1′, inside of R1′, inside of R2, inside of R2′) when there is a cleavage site inside the recognition sequence. On the other hand, when the recognition sequence and the cleavage site are distant, for example, when the first restriction enzyme cleaves at the 5′-side of R1, there will be R1 between D(iii) and D(i) ligated in step d1 described later, and there will be R1′ between D(ii) and D(iv). In this case, even when the third vector is used for ligation of further DNA fragments, the treatment with the first restriction enzyme and the second restriction enzyme in step b1 cleaves the portions between D(iii) and D(i) and between D(ii) and D(iv), breaking the ligation of DNA. Therefore, in this case, the first restriction enzyme needs to cleave the 3′-side of R1 and the second restriction enzyme needs to cleave the 5′-side of R1′, and the third restriction enzyme needs to cleave the 5′-side of R2 and a fourth restriction enzyme needs to cleave the 3′-side of R2′.

In the first method of the present invention, the protruding end of R1 cleaved with the first restriction enzyme and the protruding end of R2 cleaved with the third restriction enzyme, and the protruding end of R1′ cleaved with the second restriction enzyme and the protruding end of R2′ cleaved with the fourth restriction enzyme need to be ligatable by the ligation reaction in step d1. From this point of view, the first restriction enzyme and the third restriction enzyme, and the second restriction enzyme and the fourth restriction enzyme used are preferably two types of IIS restriction enzymes or two types of restriction enzymes that produce homologous protruding ends by DNA cleavage.

The “type IIS restriction enzyme” is a restriction enzyme in which the recognition sequence and the cleavage site are distant, and the sequence of the cleavage sites is generally any. In the first method of the present invention, in the case of using type IIS restriction enzymes, the base sequence of R1 is set so that one of the type IIS restriction enzymes recognizes R1 to cleave the 3′-side thereof, and the base sequence of R2 is set so that the other of the type IIS restriction enzymes recognizes R2 to cleave the 5′-side thereof. Similarly, the base sequence of R1′ is set so that one of the type IIS restriction enzymes recognizes R1′ to cleave the 5′-side thereof, and the base sequence of R2′ is set so that the other of the type IIS restriction enzymes recognizes R2′ to cleave the 3′-side thereof. Furthermore, a base sequence homologous to the cleavage sites is set so that the protruding ends of the two types of type IIS restriction enzymes can be ligated. The type IIS restriction enzyme used in the first method of the present invention is not particularly limited as long as the size of the protruding end becomes the same by DNA cleavage in the combination of the first restriction enzyme and the third restriction enzyme and the combination of the second restriction enzyme and the fourth restriction enzyme, and examples thereof include BsaI, BbsI, BsmBI, and BsmAI.

In the first method of the present invention, in the case of using two types of restriction enzymes that produce homologous protruding ends by DNA cleavage, one restriction enzyme recognizes R1 and cleaves its inside, and the other restriction enzyme recognizes R2 and cleaves its inside, and the protruding ends resulting from cleavage of R1 and R2 are homologous and thus ligatable to each other. Examples of the two restriction enzymes that produce homologous protruding ends by DNA cleavage used in the first method of the present invention include the combination of NheI and SpeI, the combination of AgeI and XmaI, and the combination of SalI and XhoI, but are not limited to the above as long as the object of the present invention is met.

-Selectable Marker Gene-

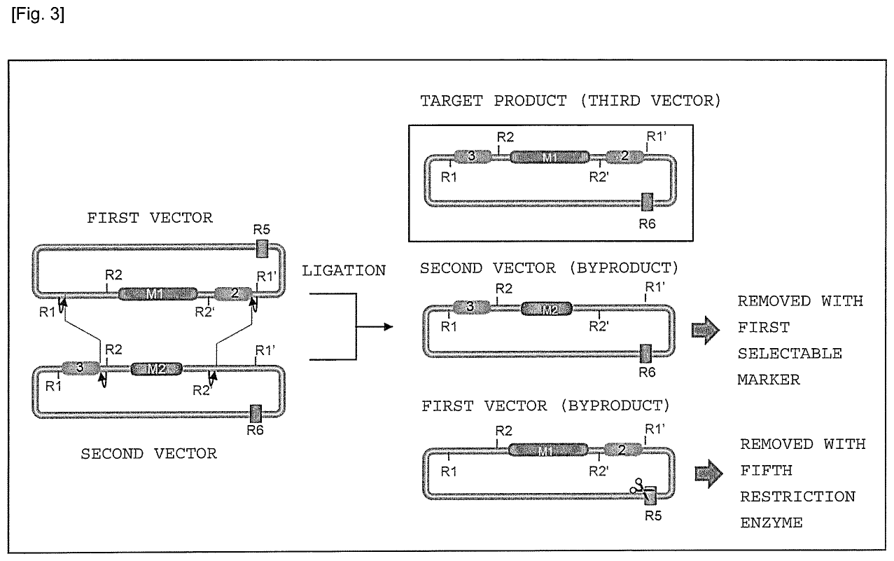

In the present invention, M1 represents the first selectable marker gene and M2 represents the second selectable marker gene. In the first method of the present invention, the first selectable marker gene is used after step d1 for the purpose of excluding non-target vectors (by-products) having the second selectable marker gene to select the target vector (third vector) having the first selectable marker gene (FIG. 3). From this point of view, the first selectable marker gene needs to be a selectable marker gene different from the second selectable marker gene.

The selectable marker gene is not particularly limited as long as it can be detected, and examples thereof include, but are not limited to, drug resistance genes, reporter genes, and counterselectable marker genes.

Examples of the drug resistance genes include spectinomycin resistance gene, ampicillin resistance gene, and chloramphenicol resistance gene. Examples of the reporter genes include green fluorescent protein (GFP), DsRed, mCherry, mOrange, mBanana, mStrawberry, mRaspberry, and mPlum. A counterselectable marker gene is a gene that causes a transformant to die when a vector having the gene is present in the transformant, and examples thereof include toxin genes such as the ccdB gene (E. coli DNA gyrase inhibitory protein (control of cell death) gene).

As to the combination of the first selectable marker gene and the second selectable marker gene, the first selectable marker gene and the second selectable marker gene are preferably the drug resistance genes from the viewpoint that the target vector can be efficiently selected using the survival of the transformant as an index.

(Step b1, Step c1)

In the first method of the present invention, the first vector is then treated with the first restriction enzyme and the second restriction enzyme to obtain a first vector fragment composed of the structure: 5′-D(i)-R2-M1-R2′-D(ii)-3′ (step b1). Meanwhile, the second vector is treated with the third restriction enzyme and the fourth restriction enzyme to obtain a second vector fragment with the removed structure: 5′-R2-M2-R2′-3′ (step c1). Either step b1 or step c1 may be performed first, or may be performed concurrently.

The restriction enzyme treatment in step b1 can be performed by allowing restriction enzymes (first restriction enzyme and second restriction enzyme) to act on the first vector in a buffer solution. When the first restriction enzyme and the second restriction enzyme in step b1 are different, either restriction enzyme treatment may be performed first, or both restriction enzymes may be added to the reaction system and treated simultaneously.

Similarly, the restriction enzyme treatment in step c1 can be performed by allowing restriction enzymes (third restriction enzyme and fourth restriction enzyme) to act on the second vector in a buffer solution. When the third restriction enzyme and the fourth restriction enzyme in step c1 are different, either restriction enzyme treatment may be performed first, or both restriction enzymes may be added to the reaction system and treated simultaneously.

As the buffer solution used in the reaction systems of step b1 and step c1, conventionally known reaction solvents for restriction enzymes may be used as appropriate, and commercially available ones such as CutSmart Buffer (NEB) may also be used as appropriate. In addition, the conditions for the reaction system can be appropriately adjusted according to the type of restriction enzyme, and for example, for 5 to 10 μg/50 μL of vector, the concentration of each restriction enzyme added to the reaction system is preferably 0.1 to 0.2 units/μL, and the concentration of each vector is preferably 100 to 200 ng/μL. Furthermore, the reaction temperature of the reaction system is preferably about 37° C., and the reaction time is preferably 1 to 2 hours.

In step b1, after restriction enzyme treatment, dephosphorylation treatment with alkaline phosphatase (such as CIP) may be performed in order to prevent self-ligation.

Moreover, step b1 can include an operation of recovering the generated first vector fragment from the reaction product, and step c1 can include an operation of recovering the generated second vector fragment from the reaction product. Such vector fragments can be recovered by size fractionation by electrophoresis such as agarose gel electrophoresis.

(Step d1)

In the first method of the present invention, the first vector fragment obtained in step b1 and the second vector fragment obtained in step c1 are then ligated by a ligation reaction to generate a third vector containing the following structure (3) (step d1):

5′-R1-D(i)₁-R2-M1-R2′-D(ii)₁-R1′-3′ (3)

Here, D(i)₁ represents a DNA fragment containing the following structure: 5′-D(iii)-D(i)-3′, and D(ii)₁ represents a DNA fragment containing the following structure: 5′-D(ii)-D(iv)-3′. Hereinafter, unless otherwise specified, the subscripts attached to D(i) to D(iv) indicate the number of times the DNA fragments have been ligated.

The ligation reaction in step d1 is a reaction for ligating the first vector fragment and the second vector fragment, and can be performed by allowing DNA ligase to act in a buffer solution. Examples of the buffer solution used in the reaction system of step d1 include the same ones as described above. Examples of DNA ligase added to the reaction system include, but are not limited to, T4 ligase. In addition, as the reaction system, the conditions can be appropriately adjusted according to the type of DNA ligase and the like, and for example, the concentration of DNA ligase added to the reaction system is preferably 20 to 40 units/μL, and the concentration of each vector fragment is preferably 100 to 200 ng/μL. Furthermore, the reaction temperature of the reaction system is preferably 16 to 25° C., and the reaction time is preferably 1 to 12 hours.

<Second Method for Producing Ligated DNA>

The second method for producing a ligated DNA of the present invention includes:
(a2) a step a2 of preparing a first vector containing the following structure (1) and a second vector containing the following structure (2):

5′-R1-D(i)-R2-M1-R2′-D(ii)-R1′-3′ (1)

5′-R1-D(iii)-R2-M2-R2′-D(iv)-R1′-3′ (2)

[Here, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one. The first restriction enzyme cleaves inside of R1 or a 3′-side of R1, and the second restriction enzyme cleaves inside of R1′ or a of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different.];
(b2) a step b2 of treating the second vector with the first restriction enzyme and the second restriction enzyme to obtain a second vector fragment composed of the structure: 5′-D(iii)-R2-M2-R2′-D(iv)-3′;
(c2) a step c2 of treating the first vector with the third restriction enzyme and the fourth restriction enzyme to obtain a first vector fragment with the removed structure: 5′-R2-M1-R2′-3′; and
(d2) a step d2 of ligating the second vector fragment obtained in step b2 and the first vector fragment obtained in step c2 by a ligation reaction to generate a fourth vector containing the following structure (4):

5′-R1-D(iii)₁-R2-M2-R2′-D(iv)₁-R1′-3′ (4)

[Here, D(iii) 1 represents a DNA fragment containing the following structure: 5′-D(i)-D(iii)-3′, and D(iv)i represents a DNA fragment containing the following structure: 5′-D(iv)-D(ii)-3′.].

As described above, the first method of the present invention includes a step of treating the first vector with a first restriction enzyme and a second restriction enzyme to replace the resulting DNA fragment “5′-D(i)-R2-M1-R2′-D(ii)-3′” with “5′-R2-M2-R2′-3′” in the second vector, and based on the same principle, the second method of the present invention treats the second vector with a first restriction enzyme and a second restriction enzyme to replace the resulting DNA fragment “5′-D(iii)-R2-M2-R2′-D(iv)-3′” with “5′-R2-M1-R2′-3′” in the first vector. Thus, unlike the first method of the invention, the second method of the invention generates a vector containing a second selectable marker gene.

(Step a2)

Step a2 in the second method of the present invention is the same as step a1 in the first method. In addition, DNA fragments for ligation, restriction enzymes and recognition sequences thereof, selectable marker genes, and preferred aspects thereof are also as described in step a1 in the first method.

(Step b2, Step c2)

Contrary to the first method, in the second method of the present invention, the second vector is then treated with a first restriction enzyme and a second restriction enzyme to obtain a second vector fragment composed of the structure: 5′-D(iii)-R2-M2-R2′-D(iv)-3′ (step b2). Meanwhile, the first vector is treated with a third restriction enzyme and a fourth restriction enzyme to obtain a first vector fragment with the removed structure: 5′-R2-M1-R2′-3′ (step c2). Either step b2 or step c2 may be performed first, or may be performed concurrently.

The restriction enzyme treatment in step b2 and the restriction enzyme treatment in step c2 are the same as the restriction enzyme treatment in step b1 and the restriction enzyme treatment in step c1, respectively, including preferred embodiments thereof. In addition, the steps of treating with alkaline phosphatase and recovering vector fragments may be further included.

(Step d2)

In the second method of the present invention, the second vector fragment obtained in step b2 and the first vector fragment obtained in step c2 are then ligated by a ligation reaction to generate a fourth vector containing the following structure (4) (step d2):

5′-R1-D(iii)₁-R2-M2-R2′-D(iv)₁-R1′-3′ (4)

Here, D(iii)₁ represents a DNA fragment containing the following structure: 5′-D(i)-D(iii)-3′, and D(iv)₁ represents a DNA fragment containing the following structure: 5′-D(iv)-D(ii)-3′. The ligation reaction in step d2 is the same as the ligation reaction in step d1, including preferred embodiments thereof.

(Transformation)

The first method of the present invention can further include, after step d1, a step of transforming a ligation reaction product into a host, and a step of using expression of the first selectable marker gene as an index to select a host introduced with the third vector. Similarly, the second method of the present invention can further include, after step d2, a step of transforming the ligation reaction product into a host, and a step of using the expression of the second selectable marker gene as an index to select a host introduced with the fourth vector.

Transformation of a ligation reaction product into a host can be performed by methods known to those skilled in the art, such as heat shock method and electroporation method. The method for selecting the host introduced with the third vector or fourth vector differs depending on the type of the first selectable marker gene or second selectable marker gene, respectively. For example, when the selectable marker gene is a drug resistance gene, survival in an environment containing the drug can be used as an indicator for selection, and when the selectable marker gene is a reporter gene, it can be selected using reporter activity (such as fluorescence) as an index.

(Fifth Restriction Enzyme, Sixth Restriction Enzyme)

In the first method and the second method of the present invention, a recognition sequence of a fifth restriction enzyme different from any of R1, R1′, R2, and R2′ (the recognition sequence is indicated by “R5” in FIG. 3) can be further set at a site other than the structure (1) in the first vector, and a recognition sequence of a sixth restriction enzyme different from any of R1, R1′, R2, R2′, and the recognition sequence of the restriction enzyme (the recognition sequence is indicated by “R6” in FIG. 3) can be further set at a site other than the structure (2) in the second vector.

The fifth and sixth restriction enzymes are not particularly limited, but preferably I-CeuI and I-SceI, which have long recognition sequences and are less likely to cause non-specific cleavage.

In this case, in step b1 in the first method of the present invention, the operation of recovering the generated first vector fragment from the reaction product can be omitted, and in step c1, the operation of recovering the generated second vector fragment from the reaction product can be omitted. That is, if the reaction product of step b1 and the reaction product of step c1 are subjected to a ligation reaction as they are, self-ligation occurs as a side reaction in which a fragment excised by restriction enzyme treatment returns to the original vector, and the original vector is produced as a by-product (FIG. 3). Even in this case, simultaneously with step b1, after step b1, or after step d1 in the first method of the present invention, the original first vector can be cleaved and removed by treatment with a fifth restriction enzyme. Meanwhile, the original second vector does not have the first selectable marker gene, and thus can be removed by selection treatment using the first selectable marker.

Similarly, when treatment with a sixth restriction enzyme is performed simultaneously with step b2 or after step b2 or after step d2 in the second method of the present invention, the original second vector can be cleaved and removed, and the original first vector does not have the second selectable marker gene, and thus can be removed by selection treatment using the second selectable marker.

(Removal of Selectable Marker Gene)

In addition, the first method of the present invention can further include, after step d1, a step of treating the third vector with the third restriction enzyme and the fourth restriction enzyme to remove the structure: 5′-R2-M1-R2′-3′ and performing a self-ligation reaction, thereby generating a fifth vector containing the structure: 5F-R1-D(i)₁-D(ii)i-R1′-3′. This allows the DNA fragments for ligation on both sides of the first selectable marker gene to be ligated. In this case, it is necessary that the third restriction enzyme and the fourth restriction enzyme are the same restriction enzymes or restriction enzymes that produce homologous protruding ends.

Similarly, the second method of the present invention can further include, after step d2, a step of treating the fourth vector with a third restriction enzyme and a fourth restriction enzyme to remove the structure: 5′-R2-M2-R2′-3′ and performing a self-ligation reaction, thereby generating a sixth vector containing the structure: 5′-R1-D(iii)₁-D(iv)₁-R1′-3′. This allows the DNA fragments for ligation on both sides of the second selectable marker gene to be ligated. In this case as well, it is necessary that the third restriction enzyme and the fourth restriction enzyme are the same restriction enzymes or restriction enzymes that produce homologous protruding ends.

The method and conditions for treatment with restriction enzymes and ligation reaction (self-ligation reaction) in the present step are the same as described above.

(Third Selectable Marker Gene, Fourth Selectable Marker Gene)

In addition, in the first method of the present invention, in order to facilitate removal of the first selectable marker gene and selection of the fifth vector generated by self-ligation, a third selectable marker gene, which is a selectable marker gene having an opposite action to that of the first selectable marker gene, is preferably further inserted between R2 and R2′ of the first vector. Similarly, in the second method of the present invention, in order to facilitate removal of the second selectable marker gene and selection of the sixth vector generated by self-ligation, a fourth selectable marker gene, which is a selectable marker gene having an opposite action to that of the second selectable marker gene, is preferably further inserted between R2 and R2′ of the second vector. In the case of using both the third selectable marker gene and the fourth selectable marker gene, the third selectable marker gene and the fourth selectable marker gene may be the same or different.

As a result, in the case of removing the above selectable marker gene, when preparing a transformant using the reaction product of the self-ligation, the third selectable marker gene or the fourth selectable marker gene is also removed, so that the expression of the third selectable marker gene or the fourth selectable marker gene can be used as an index to select the fifth vector or the sixth vector.

Here, a selectable marker gene having the opposite action to that of an any selectable marker refers to, for example, a gene whose expression renders transformants unviable when expression of the selectable marker gene allows transformants to survive. For example, when the first selectable marker gene is the drug resistance gene, the above-described counterselectable marker gene can be selected as the third selectable marker gene. In this case, for example, in steps a1 to d1 in the first method of the present invention and/or steps a2 to d2 in the second method of the present invention, when using a transformant, a host resistant to the counterselectable marker is used so that the transformant will not die.

<Repeated Ligation>

According to the first method of the present invention, DNA fragments for ligation can be sequentially ligated by repeating the cycle of steps a1 to d1. That is, the present invention provides a method for producing a ligated DNA, including a step of, after performing the first method of the present invention for one cycle, using the third vector generated in step d1 as the first vector in step a1 and repeating steps a1 to d1 for an additional n cycles (1+n cycles in total) to generate a third′ vector containing the structure (3′):

5′-R1-D(i)₁₊ₙ-R2-M1-R2′-D(ii)₁₊ₙ-R1′-3′ (3′)

Here, D(i)₁₊ₙ is the ligated DNA fragment on the 5′-slide of the first selectable marker gene obtained at cycle 1+n. In the ligated DNA fragment, D(iii) derived from the second vector is ligated to the 5′-side each time the cycle is repeated. Therefore, D(i)₁₊ₙ becomes a DNA fragment containing the structure: 5′-D(iii)-D(i)ₙ-3′. D(i)ₙ is the DNA fragment obtained at cycle n containing the structure: 5′-D(iii)-D(i)ₙ₋₁-3′, and so on.

Similarly, D(ii)₁₊ₙ is the ligated DNA fragment on the 3′-side of the first selectable marker gene obtained at cycle 1+n. In the ligated DNA fragment, D(iv) derived from the second vector is ligated to the 3′-side each time the cycle is repeated. Therefore, D(ii)₁₊ₙ becomes a DNA fragment containing the structure: 5′-D(ii)ₙ-D(iv)-3′. D(ii) n is the DNA fragment obtained at cycle n containing the structure: and so on.

Note that as described above, the subscripts attached to D(i) to D(iv) indicate the number of times the DNA fragments have been ligated (the number of cycles). For example, in any of or all of the cycles, when D(i) and D(ii) are either one and/or when D(iii) and D(iv) are either one, the number indicated by the subscript does not correspond to the number of ligated DNA fragments.

Also, between the cycles, D(iii) of the second vector may be the same or different from each other, and between the cycles, D(iv) of the second vector may be the same or different from each other. Therefore, it is possible to ligate new DNA fragments D(iii) and D(iv) on both sides of the first selectable marker gene each time the cycle is repeated.

Similarly, according to the second method of the present invention, DNA fragments for ligation can be sequentially ligated by repeating the cycle of steps a2 to d2. That is, the present invention provides a method for producing a ligated DNA, including a step of, after performing the second method of the present invention for one cycle, using the fourth vector generated in step d2 as the second vector in step a2 and repeating steps a2 to d2 for an additional n cycles (1+n cycles in total) to generate a fourth′ vector containing the structure (4′):

5′-R1-D(iii)₁₊ₙ-R2-M2-R2′-D(iv)₁₊ₙ-R1′-3′ (4′)

Here, D(iii)₁₊ₙ is the ligated DNA fragment on the 5′-slide of the second selectable marker gene obtained at cycle 1+n. In the ligated DNA fragment, D(i) derived from the first vector is ligated to the 5′-side each time the cycle is repeated. Therefore, D(iii)₁₊ₙ becomes a DNA fragment containing the structure: 5′-D(i)-D(iii)ₙ-3′. D(iii) n is the DNA fragment obtained at cycle n containing the structure: 5′-D(i)-D(iii)ₙ₋₁-3′, and so on.

Similarly, D(iv)₁₊ₙ is the ligated DNA fragment on the 3′-side of the second selectable marker gene obtained at cycle 1+n. In the ligated DNA fragment, D(ii) derived from the first vector is ligated to the 3′-side each time the cycle is repeated. Therefore, D(iv)₁₊ₙ becomes a DNA fragment containing the structure: 5′-D(iv)ₙ-D(ii)-3′. D(iv)ₙ is the DNA fragment obtained at cycle n containing the structure: 5′-D(iv)ₙ₋₁-D(ii)-3′, and so on.

Also, between the cycles, D(i) of the first vector may be the same or different from each other, and between the cycles, D(ii) of the first vector may be the same or different from each other. Therefore, it is possible to ligate new DNA fragments D(i) and D(ii) on both sides of the second selectable marker gene each time this cycle is repeated. In the first method and the second method, n is a natural number of 1 or more, and its upper limit is not particularly limited as long as the size of the ligated DNA is allowed by the vector or host cell.

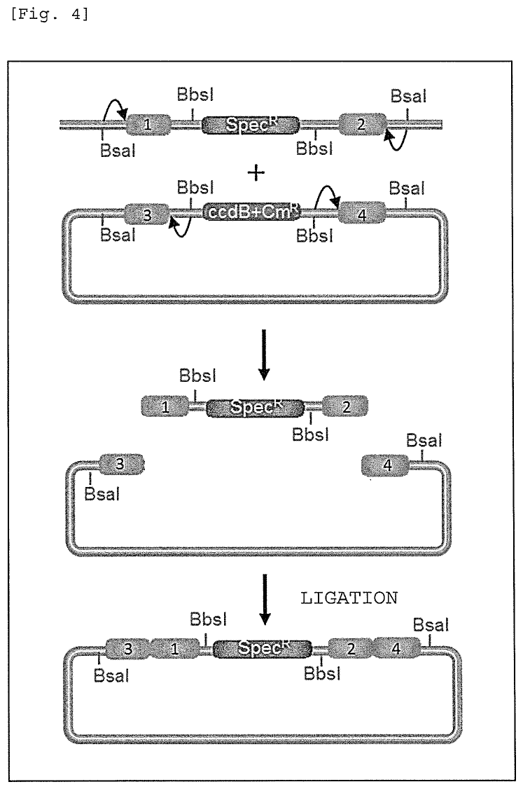

An aspect of the first method of the present invention is specifically described below with reference to FIG. 4. In the first method of the present invention, two vectors with different selectable marker genes are used (step a1). In the example of FIG. 4, the first selectable marker gene is the spectinomycin resistance gene (Spec^{R}), the second selectable marker gene is the chloramphenicol resistance gene (Cm^{R}), and the third selectable marker gene is the ccdB gene (counterselectable marker gene). BsaI is used as the first restriction enzyme and the second restriction enzyme, and BbsI is used as the third restriction enzyme and the fourth restriction enzyme. In the first vector and the second vector, each recognition sequence is placed so that BsaI as the first restriction enzyme cleaves the 3′-side of the recognition sequence R1, and BsaI as a second restriction enzyme cleaves the 5′-side of the recognition sequence R1′, and each recognition sequence is placed so that BbsI as the third restriction enzyme cleaves the 5′-side of the recognition sequence R2, and BbsI as the fourth restriction enzyme cleaves the 3′-side of the recognition sequence R2′.

In the present example, the first selectable marker gene Spec^{R} of the first vector is excised with BsaI together with DNA 1 for ligation ((D(i): 1 in FIG. 4) and DNA 2 for ligation (D(ii): 2 in FIG. 4) (step b1), and meanwhile in the second vector, the second selectable marker gene (and the third selectable marker gene) ccdB+Cm^{R} is excised and removed with BbsI (step c1), and a ligation reaction is performed so that the DNA fragment excised from the first vector (first selectable marker gene cassette) replaces the DNA fragment excised from the second vector (second selectable marker gene cassette) (step d1). Thus, a ligated DNA fragment (DNA 3 (D(iii): 3 in FIG. 4)+DNA 1) is formed on the 5′-side of the first selectable marker gene, and a ligated DNA fragment (DNA 2+DNA 4 (D(iv): 4 in FIG. 4)) is formed on the 3′-side thereof.

At this time, the BsaI recognition sequences R1 and R1′ used for cleavage in the first vector and the BbsI recognition sequences R2 and R2′ used for cleavage in the second vector do not remain in the ligated DNA fragments (between DNA 3 and DNA 1 and between DNA 2 and DNA 4). In this way, the DNA fragments can be ligated together without leaving extra recognition sequences that would cancel the ligation of the DNA fragments by the restriction enzyme treatment in the next cycle. Meanwhile, since the BsaI recognition sequence derived from the second vector and the BbsI recognition sequence derived from the first vector are left, which are not used for cleavage, in the third vector generated by the ligation reaction, at both ends of the first selectable marker gene Spec^{R}, the BbsI recognition sequence R2 and the BsaI recognition sequence R2′ are restored at the same positions as in the original first vector. Therefore, this cycle (steps a1 to d1) can be repeated many times.

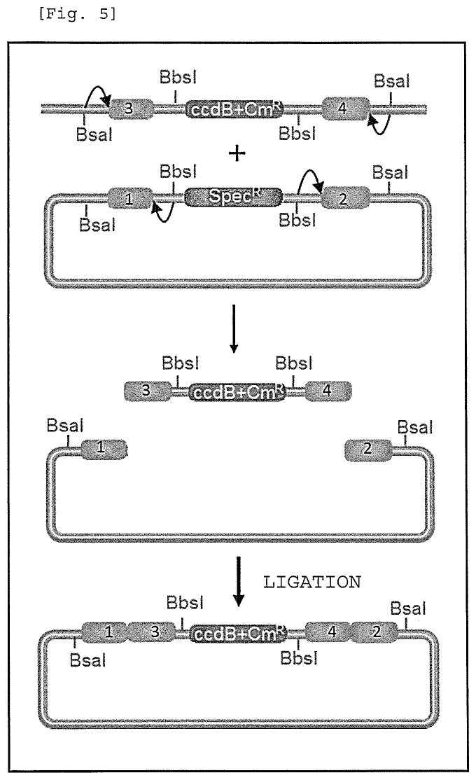

Also in the second method, the cycle (steps a2 to d2) can be repeated many times based on the same principle (FIG. 5).

<Combination of First Method and Second Method>

In the present invention, a third vector or third′ vector produced by the first method can be combined with a fourth vector or fourth′ vector produced by the second method to perform similar ligation cycles of DNA fragments.

Therefore, the present invention provides a method for producing a ligated DNA including using the third vector or third′ vector generated in step d1 in the first method as the first vector in step a2 in the second method. In addition, the present invention provides a method for producing a ligated DNA including using the fourth vector or fourth′ vector generated in step d2 in the second method as the second vector in step a1 in the first method.

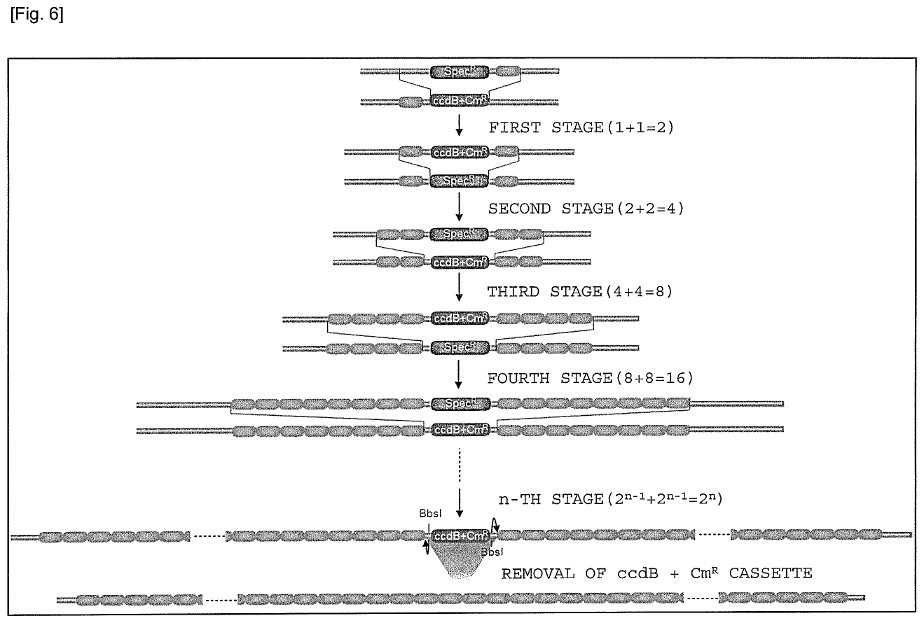

An aspect of the combinations of the present invention is described below with reference to FIG. 6. That is, in FIG. 6, first, from the first vector (vector containing Spec^{R}) and the second vector (vector containing Cm^{R}) each containing one DNA fragment for ligation, the first method of the present invention is performed for one cycle (first stage) to generate a third vector (vector containing Spec^{R}) containing two DNA fragments for ligation. In addition, similarly, from the first vector (vector containing Spec^{R}) and the second vector (vector containing Cm^{R}) each containing one DNA fragment for ligation, the second method of the present invention is performed for one cycle (first stage) to generate a fourth vector (vector containing Cm^{R}) containing two DNA fragments for ligation. These are used respectively as the first vector and the second vector in step a2 of the second method (second stage). By repeating this, the number of DNA fragments accumulated after each repetition can be increased exponentially to 1, 2, 4, 8, 16, and 32. In addition, when ligating DNA fragments, the selectable marker gene can be switched as Spec^{R} (first selectable marker gene)→Cm^{R} (second selectable marker gene)→Spec^{R}→Cm^{R}→ . . . , so that mere selection with a chemical (spectinomycin or chloramphenicol) in each cycle makes it possible to efficiently select only transformants retaining a vector introduced with the target ligated DNA with a high probability without quality inspection of the generated DNA product.

A vector (target product) retaining the target ligated DNA is preferably a vector containing a third selectable marker gene or a fourth selectable marker gene. In FIG. 6, it has the ccdB gene, which is a counter-selectable marker gene, as the fourth selectable marker gene of the second vector. E. coli strains commonly used for transformation, such as NEB5α, cannot grow and die if they carry the ccdB gene. Therefore, after cleavage by the third restriction enzyme and the fourth restriction enzyme, if self-ligation reaction is performed to transform into a host that is not ccdB resistant, a DNA product that does not have the ccdB gene in the second vector, that is, a selectable marker gene is removed, making it possible to select a DNA product ligated as a single unit. Note that when the ccdB gene is used as a counterselectable marker gene, ccdB-resistant strains are used for repeated cycles.

Further, if the recognition sequence of the fifth restriction enzyme (such as I-CeuI) is further set at a site other than the structure (1) in the first vector, and the recognition sequence of the sixth restriction enzyme (such as I-SceI) different from the recognition sequence of the fifth restriction enzyme is further set at a site other than the structure (2) in the second vector, when the selectable marker gene in the vector generated in each cycle is switched as Spec^{R} (first selectable marker gene)→Cm^{R} (second selectable marker gene)→Spec^{R}→Cm^{R}→ . . . the recognition sequence of the restriction enzyme contained in the target vector is switched accordingly as I-CeuI→I-SeuI→I-CeuI→I-SceI→ . . . Therefore, in this case, by treating the generated product with a restriction enzyme (homing nucleases) in the order I-SeuI→I-CeuI→I-SceI→I-CeuI→ . . . , non-target vectors (self-ligated by-products) can be cleaved and removed (see FIG. 3).

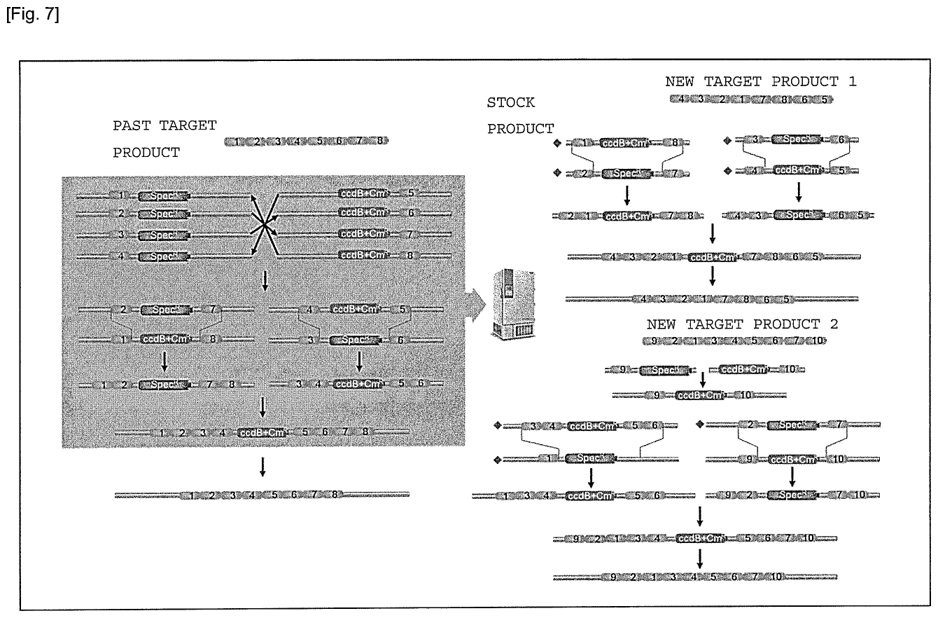

In addition, each vector as a target product or intermediate product obtained in each cycle of the first method and the second method of the present invention can be combined with each vector as a target product or intermediate product of another combination, and reused for the production of further various ligated DNAs. As the stock of various reusable products increases in this way, the number of steps required to produce a new target product decreases, thus shortening the production time and streamlining the production process (FIG. 7).

<Vector Combinations>

The present invention provides the following vector combination (first vector combination) for use in the first method and/or the second method of the present invention, that is, a first vector comprising the following structure (1) and a second vector comprising the following structure (2):

5′-R1-D(i)-R2-M1-R2′-D(ii)-R1′-3′ (1)

5′-R1-D(iii)-R2-M2-R2′-D(iv)-R1′-3′ (2)

Here, D(i) to D(iv), R1, R1′, R2, R2′, M1, and M2 are each as described above as the first vector and the second vector according to the present invention, including preferred embodiments thereof.

In addition, the present invention also provides the following vector combination (second vector combination) having DNA fragment insertion sites (sites for inserting the DNA fragment for ligation) to prepare the first vector combination, that is, a first vector comprising the following structure (1′) and a second vector comprising the following structure (2′):

5′-R1-E1-R2-M1-R2′-E2-R1′-3′ (1′)

5′-R1-E3-R2-M2-R2′-E4-R1′-3′. (2′)

Here, R1, R1′, R2, R2′, M1, and M2 are each as described above for the first vector and the second vector according to the present invention, including preferred embodiments thereof. E1, E2, E3, and E4 each independently represent a DNA fragment insertion site, and E1 and E2 may be either one, and E3 and E4 may be either one. Examples of the insertion site include, but are not limited to, a multicloning site.

Each of these vector combinations may be a combination of vectors or a kit containing the combination of vectors. A kit may further contain enzymes, buffer solutions, dilution buffer solutions, and the like necessary for each restriction enzyme reaction and ligation reaction, but is not limited to these.

<Application Example (Preparation of Genome Editing System)>

The method of the present invention is a method capable of ligating DNA fragments in a number of combination patterns by combining the first method and the second method, and by randomly combining repetition patterns and cycle counts (FRACTAL assembly method). Therefore, it can be used in various techniques regardless of the type and number of DNA fragments.

For example, when the present invention is used to prepare a genome editing system, examples of each DNA fragment for ligation D(i) to D(iv) employed include DNAs encoding repeating units of genome editing enzymes such as ZF (Zinc Finger), TALE (Transcription Activator Like Effectors), and PPR (Pentatricopeptide Repeat); and DNAs encoding guide RNAs for CRISPR-Cas (Clustered Regularly Interspaced Short Palindromic Repeats CRISPR-Associated Proteins). Further, for example, if D(i) and D(iii) employed are DNAs encoding repeating units of genome editing enzymes or guide RNAs, and D(ii) and D(iv) employed are short barcode sequences (identification sequences) specific to D(i) and D(iii), respectively, the order of ligating DNAs encoding the repeating units and guide RNAs can be determined using the ligated barcode sequences as an index without confirming all of these sequences. Specific embodiments are described below as examples.

-Ligation of gRNA-

Genome editing technology using the CRISPR-Cas9 system has rapidly spread due to its simplicity and high editing efficiency, and is now one of the standard techniques in genetic engineering. Once synthesizing a guide RNA complementary to any target sequence of about 20 bases adjacent to the PAM recognition sequence of a few bases, the guide RNA serves to guide Cas9 to the target sequence, and DNA double-strand breaks by Cas9 can disrupt the functions of the gene containing the target sequence. So far, various gene knockout libraries using CRISPR-Cas9 have been prepared for mammalian cells including humans and yeasts, but until now, there has been no technique for preparing multigene-deficient cells in which several tens or more of genes are simultaneously deleted. This is because it is difficult to mount a sequence encoding multiple guide RNAs (gRNA) on a single vector. There have been techniques for accumulating multiple gRNAs in a single vector using the Golden Gate method and the like, but the maximum number that can be accumulated is about 10. On the other hand, according to the method for producing a ligated DNA of the present invention, it is possible to ligate several tens or more of gRNAs. However, even when preparing a vector library in which several tens of gRNAs are simply linked as one array, the length of a single guide NA expression unit is about 350 bp including the promoter sequence, so that it is difficult to directly identify the array region in which gRNAs are linked in tandem by DNA sequencing.

In view of the above, the present example used the method for producing a ligated DNA of the present invention to prepare a vector library (gRNA-BC vector) in which gRNA was ligated to one end of a selectable marker gene and a barcode sequence (BC) corresponding to the gRNA was ligated to the other end (Example 1). Various multigene-deficient cells can be obtained by transfecting the prepared vector library into human cells. Furthermore, a gRNA array and the corresponding short DNA barcode array are accumulated correspondingly on the same DNA molecule, so that a combination of gRNAs can be identified by reading the base sequence of this DNA barcode array. In this example, a toolkit vector was used in which one end of the selectable marker gene was a restriction enzyme site of NheI or SpeI instead of BbsI or BsaI. In this case, the protruding ends of the DNA fragments treated with NheI and SpeI become homologous, so that they can be linked by ligation. After ligation, a sequence is formed that cannot be recognized by either restriction enzyme.

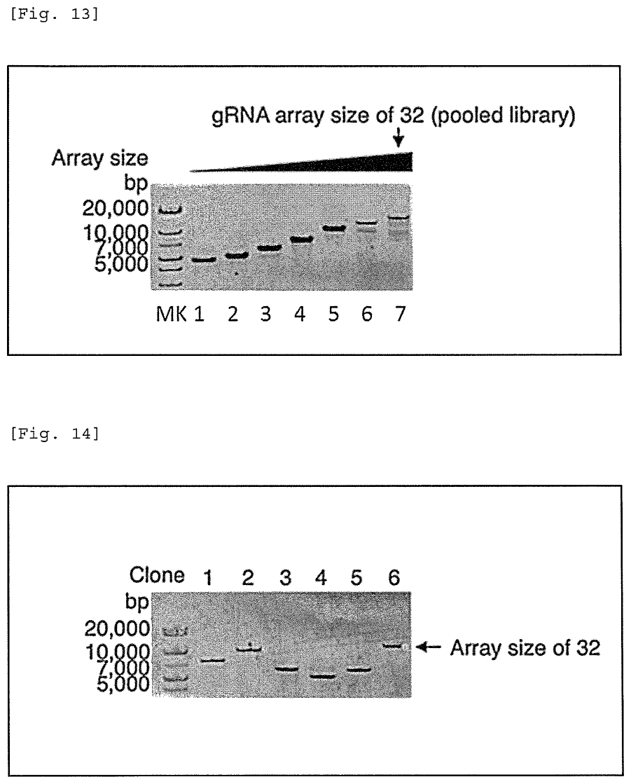

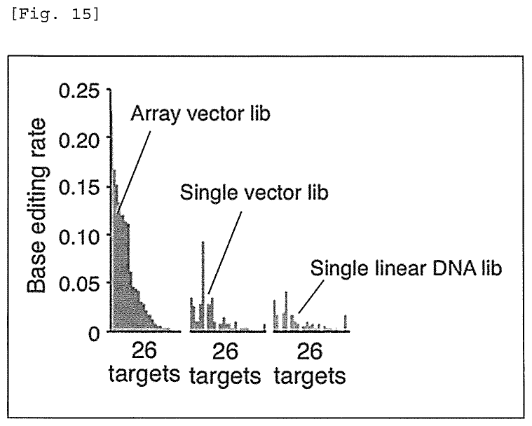

In fact, it was shown that 32 gRNAs and 32 barcode sequences corresponding to the gRNAs could be integrated into a single vector using the method for producing a ligated DNA of the present invention (FIGS. 13 and 14). Furthermore, it was shown that when a vector in which multiple gRNAs were integrated by the method of the present invention, a mixed pool of vectors containing individual gRNAs, and a mixed pool of double-stranded DNAs with individual gRNA sequences were each introduced into human cultured cells by transfection, the vector in which gRNAs were integrated had the highest genome editing efficiency (FIG. 15). In addition, as in the present toolkit vector, by inserting the Poly-A sequence outside the BsaI recognition sequence on the 3′-side of the selectable marker gene, gRNAs and barcode sequences were accumulated and then introduced into cells replacing the region of ccdB+CmR with a transcription promoter sequence, whereby the DNA barcode array was transcribed as RNA to which poly-A sequences were added. Therefore, using single-cell RNA transcriptome technology, it is possible to simultaneously read the state of each cell and the combinatorial information of their gRNAs.

Thus, if a vector capable of expressing multiple gRNAs is obtained, by constructing a CRISPR-Cas system in combination with a Cas protein, it is possible to edit DNA in multiple regions on the genome at the same time. The Cas protein to be combined may be a Cas protein with full nuclease activity, a Cas protein (nCas, dCas) in which some or all of the nuclease activity of the Cas protein has been eliminated, or a fusion protein of these Cas proteins and other enzymes. Examples of activities of other enzymes to be fused include, but are not limited to, deaminase activity (such as cystidine deaminase activity and adenosine deaminase activity), methyltransferase activity, demethylase activity, DNA repair activity, DNA damage activity, dismutase activity, alkylation activity, depurination activity, oxidation activity, pyrimidine dimer formation activity, integrase activity, transposase activity, recombinase activity, polymerase activity, ligase activity, helicase activity, photolyase activity, or glycosylase activity. The Cas protein may be a fusion protein with a transcriptional regulatory protein. Examples of transcription regulatory proteins include, but are not limited to, light-induced transcription regulatory factors, small molecule/drug-responsive transcription regulatory factors, transcription factors, transcription repressors, and the like. When preparing a fusion protein, a linker sequence may be interposed, if necessary.

-Ligation of TALE Repeat Units-

Protein sequences used for genome editing such as TALE and zinc finger have a structure in which several types of repeat unit sequences containing partially different sequences are repeated in tandem. For example, in TALE, each of 4 or 5 types of repeat unit sequences with partially different amino acid residues specifically recognizes bases. So far, a method using the Golden Gate method has been used as a general method for synthesizing TALE repeat unit arrays. However, it is necessary to prepare different fragment sequences according to the target TALE repeat unit array. Meanwhile, the method of the present invention makes it possible to produce any combination of TALE repeat unit arrays, and in particular, by dividing the TALE repeat unit and allowing variations only to fragments containing variable regions of amino acid residues (RVD: Repeat Variable Diresidue), a pool library (TALE repeat unit array) containing various TALE repeat units can be prepared by only preparing several types of other fragments (FIG. 8).

In fact, using the method for producing a ligated DNA of the present invention, TALE repeat units divided into 3 fragments were ligated to finally synthesize a TALE repeat unit array composed of 48 fragments and 16 repeats (Example 2). This method can be applied not only to TALEs but also to repeat proteins composed of several partially different types of repeat units, such as zinc fingers and PPR (pentatricopeptide repeat) proteins.

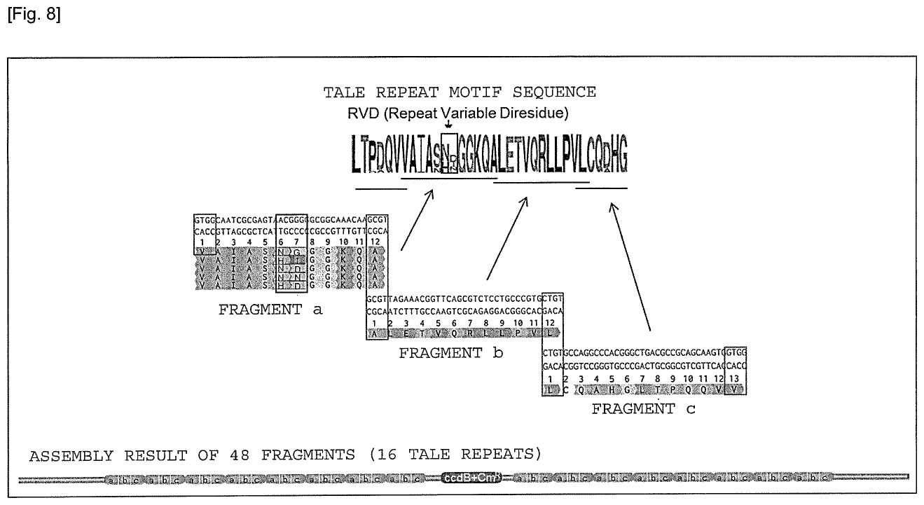

In addition, the idea of simultaneously accumulating gRNAs and barcode sequences corresponding to the gRNAs can be applied when it is desired to efficiently obtain protein repeats. For example, in the case of ligation of TALE repeat units, a vector is first prepared for each TALE repeat unit, in which one TALE repeat unit is inserted at one end of the selectable marker gene of the toolkit vector and the corresponding barcode sequence is inserted at the other end thereof. A mixture of these vectors is then ligated according to the method of the invention, resulting in a library pool of different TALE repeat unit arrays with corresponding DNA barcode arrays (FIG. 9).

After that, when a random DNA sequence of about bases is inserted into the 3′-end of the barcode array, a 30-base DNA sequence specific to each DNA molecule having the barcode array is added. A short region encompassing the 3′-end of the barcode array to the 5′-end of the random barcode can be amplified by PCR and read out simultaneously by a massively parallel DNA sequencer, so that in the library pool, the target TALE repeat unit array can be identified using the corresponding DNA barcode array as an index, and the random barcode sequence associated therewith can also be identified. Therefore, when taking out a specific TALE repeat unit array from the library pool, by using PCR primers that specifically bind to random barcode sequences corresponding to TALE repeat unit arrays, it becomes possible to amplify and extract only the target TALE repeat unit arrays from the library pool. Genome editing using TALE has a disadvantage that generating a target TALE repeat unit is more complicated than generating gRNA, but by simply using specific primers from a library pool prepared in advance in this way, it is possible to take out a target product.

### EXAMPLES

Examples of the present invention are described below, but the present invention is not limited to these Examples. Hereinafter, the plasmids used were as follows.

<Plasmid pNM1088 (Spec^{R})>

A PCR product amplified with forward primer DG012 (SEQ ID NO: 1) and reverse primer DG011 (SEQ ID NO: 2) using pUC19 (New England Biolabs Japan (NEB)) as a template; a PCR product amplified with forward primer DG009 (SEQ ID NO: 3) and reverse primer DG010 (SEQ ID NO: 4) using pUC19 (NEB) as a template; a PCR product amplified with forward primer DG007 (SEQ ID NO: 5) and reverse primer DG008 (SEQ ID NO: 6) using pLVSIN-CMV Pur Vector (Takara) as a template; a PCR product amplified with forward primer DG001 (SEQ ID NO: 7) and reverse primer DG002 (SEQ ID NO: 8) using pUC19 (NEB) as a template; and a PCR product amplified with forward primer DG003 (SEQ ID NO: 9) and reverse primer DG004 (SEQ ID NO: 10) using pINDUCER20 (addgene) as a template were prepared by ligation using Gibson Assembly. Plasmid pNM1088 (Spec^{R}) has the spectinomycin resistance gene (Spec^{R}).

<Plasmid pNM1089 (ccdB+Cm^{R})>

A PCR product amplified with forward primer DG012 and reverse primer DG011 using pUC19 (NEB) as a template; a PCR product amplified with forward primer DG009 and reverse primer DG010 using pUC19 (NEB) as a template; a PCR product amplified with forward primer DG007 and reverse primer DG008 using pLVSIN-CMV Pur Vector (Takara) as a template; a PCR product amplified with forward primer DG001 and reverse primer DG002 using pUC19 (NEB) as a template; and a PCR product amplified with forward primer DG013 (SEQ ID NO: 11) and reverse primer DG015 (SEQ ID NO: 12) using pDONR223 (addgene) as a template were prepared by ligation using Gibson Assembly. Plasmid pNM1089 (ccdB+Cm^{R}) has a combination of the chloramphenicol resistance gene (Cm^{R}) and the E. coli DNA gyrase inhibitory protein (control of cell death) gene (ccdB) (ccdB+Cm^{R}).

<Plasmid pKK1010 (Amp^{R})>

A PCR product amplified with forward primer DG021 (SEQ ID NO: 13) and reverse primer DG015 using pDONR223 (addgene) as a template; and a PCR product amplified with forward primer M13-Fw (SEQ ID NO: 14) and reverse primer DG008 using pNM1088 as a template were prepared by ligation using Gibson Assembly. Plasmid pKK1010 (Amp^{R}) has the ampicillin resistance gene (Amp^{R}).

<Plasmid pKK1009 (Amp^{R})>

A PCR product amplified with forward primer DG020 (SEQ ID NO: 15) and reverse primer DG006 (SEQ ID NO: 16) using pDONR223 (addgene) as a template; and a PCR product amplified with forward primer DG012 and reverse primer DG009 using pNM1089 as a template were prepared by ligation using Gibson Assembly. Plasmid pKK1009 (Amp^{R}) has the ampicillin resistance gene (Amp^{R}).

1. Preparation of gRNA-BC Vector (FRACTAL Assembly Method)

A guide RNA-encoding sequence (gRNA) and a corresponding barcode-encoding sequence (BC) were integrated into one vector by the method for producing a ligated DNA of the present invention (FRACTAL assembly method) to prepare a gRNA-BC vector.

1.1 Amplification of DNA Fragment Containing gRNA, BC, and Selectable Marker Gene (gRNA-BC Unit)

Forward primers 1 to 96 containing sequences (gRNAs 1 to 96) encoding guide RNAs 1 to 96 targeting the 96 gene regions of the human ABC transporter (the sequence of forward primer NM_ABC001Fw containing the sequence encoding guide RNA1 (gRNA1) is shown as an example in SEQ ID NO: 17) and reverse primers containing sequences (BC 1 to 96) encoding barcodes corresponding to the gRNAs (the sequence of reverse primer NM_ABC001Rv containing the sequence encoding the barcode corresponding to gRNA1 (BC1) is shown as an example in SEQ ID NO: 18) were used, and plasmid pNM1088 (Spec^{R}) and plasmid pNM1089 (ccdB+Cm^{R}) were used as templates for amplification by PCR. As a result, DNA fragments containing “5′-gRNA1-Spec^{R}-BC1-3′” to “5′-gRNA96-Spec^{R}-BC96-3′” (96 types in total) and DNA fragments containing “5′-gRNA1-ccdB+Cm^{R}-BC1-3′” to “5′-gRNA96-ccdB+Cm^{R}-BC96-3′” (96 types in total) were obtained (hereinafter, these 192 types of DNA fragments are collectively referred to as “gRNA-BC unit” in some cases). The NheI recognition sequence and the BsaI recognition sequence were inserted on the 5′-side and 3′-side of each DNA fragment, respectively, using the above primers, and the SpeI recognition sequence was inserted between the gRNA and each marker gene, and the BbsI recognition sequence was inserted between BC and each marker gene, respectively, using the above primers. The PCR conditions are shown below.

[PCR Conditions]
Reaction solution (Total: 20 μL):

| | |
|---|---|
| 5 × GC buffer | 4.0 μL |
| 2.5 μM dNTPs | 0.4 μL |
| Phusion DNA polymerase | 0.4 μL |
| 2 μM forward primer | 5.0 μL |
| 2 μM reverse primer | 5.0 μL |
| DMSO | 1.0 μL |
| 50 pg/μL template plasmid | 1.0 μL |
| ddH₂O | 3.2 μL |

Reaction conditions:
   1. 95° C. 30 seconds
   2 to 5. 95° C. 10 seconds, 53° C. 10 seconds, 72° C. 1 minute: 30 cycles
   6. 72° C. 5 minutes
   7. 4° C.∞.

1.2 Preparation of Toolkit Vector (Insertion of gRNA-BC Unit into Vector)

The PCR product (gRNA-BC unit) of 1.1 above was cleaved with restriction enzymes NheI (NheI-HF, NEB) and BsaI (BsaI-HFv2, NEB) to obtain a Donor DNA. In addition, as a Host DNA, plasmid pKK1010 (Amp^{R}) and plasmid pKK1009 (Amp^{R}) were cleaved with restriction enzymes SpeI (SpeI-HF, NEB) and BbsI (BbsI-HF, NEB), respectively. Note that a homologous sequence capable of ligating to the cleaved end of BbsI was inserted into the cleaved end of BsaLI using the above primers. The DNA fragments containing “5′-gRNA1-Spec^{R}-BC1-3′” to “5′-gRNA96-Spec^{R}-BC96-3′” were ligated into plasmid pKK1010 (Amp^{R}) to prepare toolkit vector 1(n) (n: 1 to 96) containing each of the DNA fragments. In addition, the DNA fragments containing “5′-gRNA1-ccdB+Cm^{R}-BC1-3′” to “5′-gRNA96-ccdB+Cm^{R}-BC96-3′” were ligated into plasmid pKK1009 (Amp^{R}) to prepare toolkit vector 2(n) (n: 1 to 96) containing each of the DNA fragments. The restriction enzyme treatment conditions and ligation conditions are shown below.

[Restriction Enzyme Treatment Conditions]

### [NheI/BsaI for Donor DNA]

Reaction solution (Total: 50 μL):

| | | |
|---|---|---|
| 10 × CutSmart Buffer | 5 | μL |
| NheI (20,000 units/mL) | 1 | μL |
| BsaI (20,000 units/ml) | 1 | μL |
| PCR product (donor vector after 1.4) | 5 | μg |

| | |
|---|---|
| ddH₂O | balance |

Reaction conditions
   1. 37° C. 2 hours
   2. 1 μL CIP was added to 50 μL reaction solution
   3. 37° C. 30 minutes

### [SpeI/BbsI for Host DNA]

Reaction solution (Total: 50 μL):

| | | |
|---|---|---|
| 10 × CutSmart Buffer | 5 | μL |
| SpeI (20,000 units/mL) | 1 | μL |
| BbsI (20,000 units/ml) | 1 | μL |
| plasmid (host vector after 1.4) | 5 | μg |

| | |
|---|---|
| ddH₂O | balance |

Reaction conditions
   1. 37° C. 2 hours.

### [Ligation Conditions]

Reaction solution (Total: 20 μL):

| | | |
|---|---|---|
| 10 × ligation buffer | 2 | μL |
| Donor DNA | 100 | ng |
| Host DNA | 100 | ng |
| T4 DNA Ligase (350 U/μL) | 1 | μL |

| | |
|---|---|
| ddH₂O | balance |

Reaction conditions
   1. 16° C. 2 hours
   2. 4° C. ∞.

1.3 Transformation of Toolkit Vector

The ligation products of 1.2 above were transformed into E. coli, and a drug-selective medium containing antibiotics corresponding to the selectable marker genes contained in the gRNA-BC unit of Donor DNA was used to select E. coli containing the desired toolkit vector. First, when the selectable marker gene was Spec^{R}, 2.5 μL of the ligation product was added to 30 μL NEB 5-alpha Competent E. coli (NEB). In addition, when the selectable marker gene was ccdB+Cm^{R}, 2.5 μL of the ligation reaction solution was added to 30 μL One Shot™ ccdB Survival™ 2 T1^{R} Competent Cells (Invitrogen). Then, these were allowed to stand on ice for 30 minutes, then incubated (heat shock) in a 42° C. water bath for 30 seconds, and then allowed to stand on ice for 2 minutes. Then, 250 μL of Soc medium was added to each of them, and after incubation at 37° C. for 2 hours, all of the incubated culture solutions were seeded on LB agar medium containing antibiotics corresponding to the selectable marker genes. When the selectable marker gene contained in the gRNA-BC unit was Spec^{R}, the antibiotics were ampicillin (Amp) and spectinomycin (Spec), and when the selectable marker gene contained in the gRNA-BC unit was ccdB+Cm^{R}, the antibiotics are ampicillin (Amp) and chloramphenicol (Cm). Then, after incubation at 16° C. for 3 to 4 days, the target toolkit vectors, that is, toolkit vector 1(n) and toolkit vector 2(n), were isolated from the E. coli whose growth was confirmed. Each of these toolkit vectors is a gRNA-BC vector containing one set of gRNA-BC units.

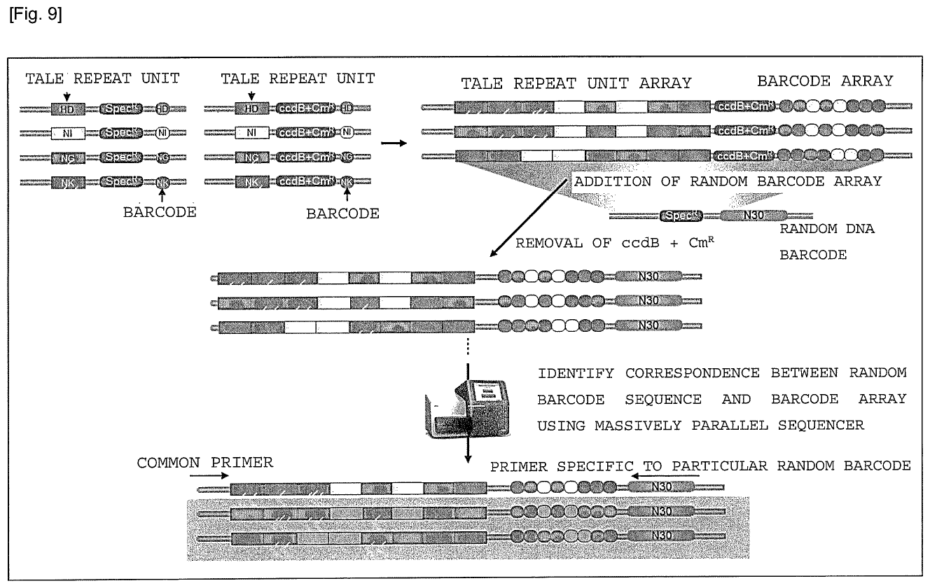

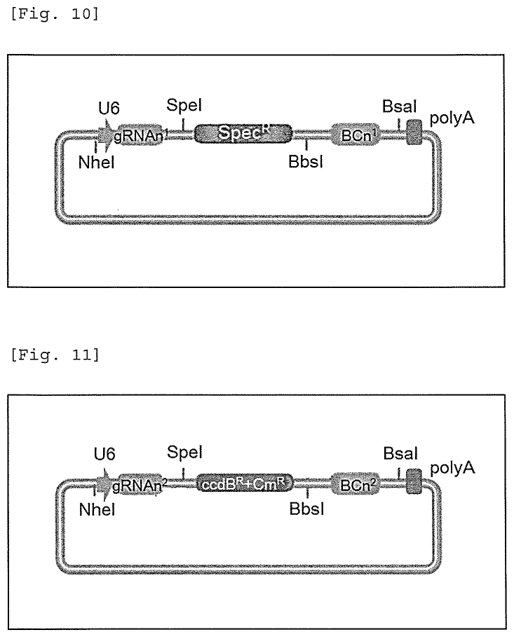

Among the obtained toolkit vector 1(n) and toolkit vector 2(n), FIG. 10 shows a schematic diagram of toolkit vector 1(n′) containing a gRNAn¹-BCn¹ unit where n is a freely-selected n¹, and FIG. 11 shows a schematic diagram of toolkit vector 2(n²) containing a gRNAn²-BCn² unit where n is a freely-selected n². The 5′-side NheI recognition sequence inserted into each DNA fragment is extinguished by the above ligation, but toolkit vector 1(n) and toolkit vector 2(n) each contain a Host DNA-derived NheI recognition sequence and U6 promoter sequence on the 5′-side of gRNA, and the poly A sequence on the 3′-side of the BsaI recognition sequence. Note that the 3′-side BsaI recognition sequence inserted into each DNA fragment is not extinguished by the above ligation (the Host DNA-derived BbsI recognition sequence is removed by the above ligation).

1.4.1 Toolkit Vector Cleaving Process 1

As donor vectors containing gRNA-BC units to be added, 96 types of toolkit vectors 2(1 to 96) (vector containing “gRNA1-ccdB+Cm^{R}-BC1” to vector containing “gRNA96-ccdB+Cm^{R}-BC96”) were mixed and cleaved with restriction enzymes NheI and BsaI. Meanwhile, as host vectors to receive the sets, 96 types of toolkit vectors 1(1 to 96) (vector containing “gRNA1-Spec^{R}-BC1” to vector containing “gRNA96-Spec^{R}-BC96”) were mixed, and cleaved with restriction enzymes SpeI and BbsI. The restriction enzyme treatment conditions are as shown in 1.2 above.

1.4.2 Linking DNA Fragments by Ligation 1 (gRNAn¹-gRNAn², BCn²-BCn¹)

A mixture of fragments (“5′-gRNAn²-ccdB+Cm^{R}-BCn²-3′”, n²: any of 1 to 96) containing one set of gRNA-BC units excised from the donor vector in 1.4.1 above and a mixture of fragments (“gRNAn¹-3′/5′-BCn¹′”, n¹: any of 1 to 96) containing one set of gRNA-BC units obtained by removing the selectable marker gene (Spec^{R}) from the host vector were collected and linked by ligation. The ligation conditions are as shown in 1.2 above.

1.4.3 Transformation of Target Vector 1

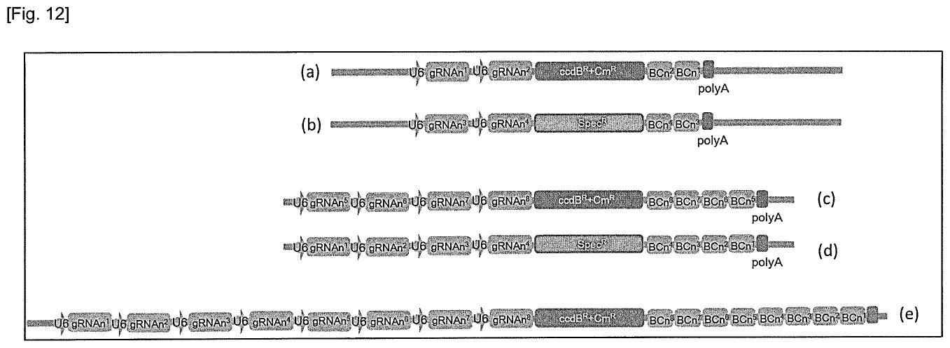

The ligation product of 1.4.2 above in an amount of 2.5 μL was added to 30 μL of One Shot™ ccdB Survival™ 2 T1^{R} Competent Cells (Invitrogen). Then, this was allowed to stand on ice for 30 minutes, then incubated (heat shock) in a 42° C. water bath for 30 seconds, and then allowed to stand on ice for 2 minutes. Then, 250 μL of Soc medium was added, and after incubation at 37° C. for 2 hours, all of the incubated culture solutions were seeded on LB agar medium containing ampicillin (Amp) and chloramphenicol (Cm). After incubation at 16° C. for 3 to 4 days, the target vector, that is, a vector containing “5′-gRNAn¹-gRNAn²-ccdB+Cm^{R}-BCn²-BCn¹-3′, n¹, n²: each independently any of 1 to 96″ (toolkit vector 2(n¹, n²)) was isolated from the E. coli whose growth was confirmed. FIG. 12(a) shows a schematic diagram of the gRNA-BC unit of the resulting toolkit vector 2 (n¹, n²).

1.5.1 Toolkit Vector Cleaving Process 2

As donor vectors containing gRNA-BC units to be added, toolkit vectors 1(1 to 96) (vector containing “gRNA1-Spec^{R}-BC1” to vector containing “gRNA96-Spec^{R}-BC96”) were mixed and cleaved with restriction enzymes NheI and BsaI. Meanwhile, as host vectors to receive the sets, toolkit vectors 2(1 to 96) (vector containing “gRNA1-ccdB+Cm^{R}-BC1” to vector containing “gRNA96-ccdB+Cm^{R}-BC96”) were mixed, and cleaved with restriction enzymes SpeI and BbsI. The restriction enzyme treatment conditions are as shown in 1.2 above. 1.5.2 Linking DNA Fragments by Ligation 2 (gRNAn³-gRNAn⁴, BCn⁴-BCn³)

A mixture of fragments (“5′-gRNAn⁴-Spec^{R}-BCn⁴-3′”, n⁴: any of 1 to 96) containing gRNA-BC units excised from the donor vector in 1.5.1 above and a mixture of fragments (“gRNAn³-3′/5′-BCn³”, n³: any of 1 to 96) containing one set of gRNA-BC units obtained by removing the selectable marker gene (ccdB+Cm^{R}) from the host vector were collected and linked by ligation. The ligation conditions are as shown in 1.2 above.

1.5.3 Transformation of Target Vector 2

The ligation product of 1.5.2 above in an amount of 2.5 μL was added to 30 μL of NEB 5-alpha Competent E. coli (NEB). Then, this was allowed to stand on ice for 30 minutes, then incubated (heat shock) in a 42° C. water bath for 30 seconds, and then allowed to stand on ice for 2 minutes. Then, 250 μL of Soc medium was added, and after incubation at 37° C. for 2 hours, all of the incubated culture solutions were seeded on LB agar medium containing ampicillin (Amp) and spectinomycin (Spec). After incubation at 16° C. for 3 to 4 days, the target vector, that is, a vector containing “5′-gRNAn³-gRNAn⁴-Spec^{R}-BCn⁴-BCn³-3′” (n³, n⁴: each independently any of 1 to 96) (toolkit vector 1(n³, n⁴)) was isolated from the E. coli whose growth was confirmed. FIG. 12(b) shows a schematic diagram of the gRNA-BC unit of the resulting toolkit vector 1(n³, n⁴). The resulting toolkit vector is a gRNA-BC vector containing 2 sets of gRNA-BC units.

1.6 Linking DNA Fragments 3 (gRNA n¹ to n⁴, BC n¹ to n⁴)

The vector, that is, a vector containing “5′-gRNAn¹-gRNAn²-gRNAn³-gRNAn⁴-Spec^{R}-BCn⁴-BCn³-BCn²-BCn¹-3′” (toolkit vector 1(n¹ to n⁴)) was obtained in the same manner as in 1.5.1 to 1.5.3 except for using a mixture of toolkit vectors 1(n³, n⁴) containing 2 sets of gRNA-BC units obtained in 1.5.3 (n³, n⁴: each independently any from 1 to 96 between vectors) instead of a mixture of toolkit vectors 1(1 to 96) as donor vectors containing the gRNA-BC unit to be added, and using a mixture of toolkit vector 2(n¹, n²) containing 2 sets of gRNA-BC units obtained in 1.4.3 above (n¹, n²: each independently any from 1 to 96 between vectors) instead of a mixture of toolkit vectors 2(1 to 96) as host vectors to receive the sets. FIG. 12(d) shows a schematic diagram of the gRNA-BC unit of the resulting toolkit vector 1(n¹ to n⁴). The resulting toolkit vector is a gRNA-BC vector containing 4 sets of gRNA-BC units.

1.7 Linking DNA Fragments 4 (gRNAn⁵ to n⁸, BCn¹ to n⁸)

The vector, that is, a vector containing “5′-gRNAn⁵-gRNAn⁶-gRNAn⁷-gRNAn⁸-ccdB+Cm^{R}-BCn⁸-BCn⁷-BCn⁶-BCn⁵-3′” (toolkit vector 2(n⁵ to n⁸)) was obtained in the same manner as in 1.4.1 to 1.4.3 except for using, as a mixture of toolkit vector 2 (n⁷, n⁸), a mixture of toolkit vectors 2(n¹, n²) containing 2 sets of gRNA-BC units obtained in 1.4.3 (n¹, n²: each independently any from 1 to 96 between vectors) instead of a mixture of toolkit vectors 2(1 to 96) as donor vectors containing the gRNA-BC unit to be added, and using, as a mixture of toolkit vector 1(n⁵, n⁶), a mixture of toolkit vector 1(n³, n⁴) obtained in 1.5.3 (n³, n⁴: each independently any from 1 to 96 between vectors) instead of a mixture of toolkit vectors 1(1 to 96) as host vectors to receive the sets. FIG. 12(c) shows a schematic diagram of the gRNA-BC unit of the resulting toolkit vector 2(n⁵ to n⁸). The resulting toolkit vector is a gRNA-BC vector containing 4 sets of gRNA-BC units.

1.8 Linking DNA Fragments 5 (gRNA n¹ to n⁸, BC n¹ to n⁸, and later)

The toolkit vectors obtained in 1.6 and 1.7 above were used to repeat 1.4.1 to 1.4.3, and to obtain a toolkit vector containing 8 sets of gRNA-BC units (gRNA-BC vector), that is, a vector containing “5′-gRNAn¹-gRNAn²-gRNAn³-gRNAn⁴-gRNAn⁵-gRNAn⁶-gRNAn⁷-gRNAn⁸-ccdB+Cm^{R}-BCn⁸-BCn⁷-BCn⁶-BCn⁵-BCn⁴-BCn³-BCn²-BCn¹-3′” (toolkit vector 2 (n¹ to n⁸)). FIG. 12(e) shows a schematic diagram of the gRNA-BC unit of the resulting toolkit vector 2 (n¹ to n⁸). In addition, the toolkit vectors obtained in 1.6 and 1.7 above were used to repeat 1.5.1 to 1.5.3, and to similarly prepare one whose selectable marker gene contained in the gRNA-BC unit was Spec^{R}.

Furthermore, the above 1.4.1 and subsequent steps were similarly repeated to prepare toolkit vectors 1 (n¹ to n¹⁶) and toolkit vectors 2 (n¹ to n¹⁶) each containing 16 sets of gRNA-BC units (gRNA-BC vectors) as well as toolkit vectors 1 (n¹ to n³²) and toolkit vectors 2 (n¹ to n³²) each containing 32 sets of gRNA-BC units (gRNA-BC vectors).

FIG. 13 shows an electropherogram of fragments obtained by using restriction enzyme SpeI to cleave plasmid pKK1009 used in 1.2 above (lane 1), toolkit vector 2(n) containing 1 set of gRNA-BC units obtained in 1.3 (lane 2), toolkit vector 2(n¹, n²) containing 2 sets of gRNA-BC units obtained in 1.4.3 (lane 3), toolkit vector 2(n⁵ to n⁸) containing 4 sets of gRNA-BC units obtained in 1.7 (lane 4), and toolkit vector 2(n¹ to n⁸) containing 8 sets of gRNA-BC units (lane 5), toolkit vector 2(n¹ to n¹⁶) containing 16 sets of gRNA-BC units (lane 6), and toolkit vector 2(n¹ to n³²) containing 32 sets of gRNA-BC units (lane 7) obtained in 1.8. As shown in FIG. 13, it was confirmed that each time the ligation was repeated, the molecular weight increased, and both gRNA and BC were accumulated stepwise (0, 1, 2, 4, 8, 16, 32 pieces).

In addition, FIG. 14 shows an electropherogram of fragments obtained by using restriction enzyme SpeI to cleave the vectors isolated from clones 1 to 6 obtained by transforming into E. coli the ligation products obtained by repeating 1.4.1 to 1.8 above so as to contain 32 sets of gRNA-BC units. As shown in FIG. 14, it was confirmed that 32 gRNAs and 32 BCs could be integrated into a single vector by the method for producing a ligated DNA of the present invention (FRACTAL assembly method) (clones 2 and 6).

1.9. Transfection of gRNA-BC Vector into HEK293Ta Cells

A genome editing assay was performed using a gRNA-BC vector containing a gRNA-BC unit obtained by the method for producing a ligated DNA of the present invention.

Specifically, first, a DNA fragment containing puromycin resistance gene was amplified by PCR from pLVSIN-CMV Pur Vector (Takara), and the amplified PCR product and a mixture of toolkit vectors 2 (n¹ to n³²) containing 32 sets of gRNA-BC units obtained in 1.8 above (n¹ to n³²: each independently any from 1 to 96 between vectors) were cleaved with restriction enzymes SpeI (SpeI-HF, NEB) and BamHI (BamHI-HF, NEB), respectively, and ligated together to prepare a mixture of vectors (array vectors) in which the selectable marker gene contained in the gRNA-BC unit had been replaced with puromycin resistance gene from ccdB+Cm^{R}. Similarly, as for each of the 96 types of toolkit vectors 2 containing 1 set of gRNA-BC units obtained in 1.3, a vector (single vector) in which the selectable marker gene ccdB+Cm^{R} had been replaced with puromycin resistance gene was prepared in the same manner. The restriction enzyme treatment conditions and ligation conditions are shown below.

[Restriction Enzyme Treatment Conditions]

### [SpeI/BamHI]

Reaction solution (Total: 50 μL):

| | | |
|---|---|---|
| 10 × CutSmart Buffer | 5 | μL |
| SpeI (20,000 units/mL) | 1 | μL |
| BamHI (20,000 units/ml) | 1 | μL |
| PCR product or toolkit vector | 5 | μg |

| | |
|---|---|
| ddH₂O | balance |

Reaction conditions for PCR product
   1. 37° C. 2 hours
   2. 1 μL CIP was added to 50 μL reaction solution
   3. 37° C. 30 minutes
Reaction conditions for toolkit vector
   1. 37° C. 2 hours.

[Ligation Conditions]
Reaction solution (Total: 20 μL):

| | | |
|---|---|---|
| 10 × ligation buffer | 2 | μL |
| PCR product | 100 | ng |
| Toolkit vector | 100 | ng |
| T4 DNA Ligase (350 U/μL) | 1 | μL |

| | |
|---|---|
| ddH₂O | balance |

Reaction conditions
   1. 16° C. 2 hours
   2. 4° C. ∞.

E. coli containing each vector was selected to isolate Array vector or Single vector in the same manner as in 1.3 above except that each ligation product obtained was transformed into E. coli (NEB 5-alpha Competent E. coli (NEB)), and the antibiotic was changed to puromycin. In the following transfection into HEK293Ta cells, as the Array vector lib, a mixture of Array vectors isolated from multiple E. coli was used, and as the Single vector lib, a mixture of 96 types of Single vectors produced from 96 types of toolkit vectors 2(n) was used. In addition, as a control, a mixture of 96 types of DNA fragments containing only each gRNA-BC unit, obtained by treating 96 types of toolkit vectors 2(n) with restriction enzymes NheI and BsaI, was used as a Single liner DNA lib.

On the day before transfection, 0.1×10⁶ cells/well of HEK293Ta cells were passaged to a 12-well plate. In addition, per well, 0.25 μg (2.5 μL) of Array vector lib, Single vector lib, or Single liner DNA lib, 0.25 μg (2.5 μL) of Target-AID vector (manufactured by Addgene), and 1.5 μL of PEI were mixed with 93.5 μL of PBS and allowed to stand at room temperature for 20 minutes. In Target-AID, when DNA is dissociated into single strands by a guide RNA, cytosine deaminase chemically replaces the bases of dissociated single-stranded DNA from cytosine (C) to thymine (T) to edit the genome. Twenty-four hours after passage, the medium was replaced, and each mixture after standing was added to the cells for transfection. Eighteen hours after transfection, the medium was replaced, and 48 hours later, 2 μg/mL puromycin was added to the medium for cell selection. Thereafter, the medium was replaced every 48 hours, and genomic DNA was extracted days after transfection.

Then, using each genomic DNA as a template, the target regions (96 sites) of the 96 types of guide RNA were amplified by PCR. Among the forward primers used for PCR, the sequence of forward primer NM_ABC_gt_1_Fw for the target sequence of guide RNA1 is set forth in SEQ ID NO: 19 as an example, and the sequence of reverse primer NM_ABC_gt_1_Rv for the target sequence of guide RNA1 is set forth in SEQ ID NO: 20 as an example. In addition, the PCR conditions are shown below.

[PCR Conditions]
Reaction solution (Total: 30 μL):

| | | |
|---|---|---|
| 5 × HF buffer | 6.0 | μL |
| 25 μM dNTPs | 2.4 | μL |
| Phusion DNA polymerase | 0.3 | μL |
| 10 μM forward primer | 3.0 | μL |
| 10 μM reverse primer | 3.0 | μL |
| 250 ng/μL genome DNA | 1.0 | μL |
| ddH₂O | 14.3 | μL |

Reaction conditions:
   1. 98° C. 10 minutes
   2 to 5. 98° C. 10 seconds, 58.4° C. 10 seconds, 72° C. 15 seconds: 25 cycles
   6. 72° C. 5 minutes
   7. 4° C. ∞.

Next, to prepare an Illumina library, the following PCR was performed using the PCR product as a template and forward primer BC_0074 (SEQ ID NO: 21) and reverse primer BC_0075 (SEQ ID NO: 22).

[PCR Conditions]
Reaction solution (Total: 30 μL):

| | | |
|---|---|---|
| 5 × GC buffer | 6.0 | μL |
| 25 μM dNTPS | 0.6 | μL |
| Phusion DNA polymerase | 0.6 | μL |
| 10 μM forward primer | 1.5 | μL |
| 10 μM reverse primer | 1.5 | μL |
| DMSO | 0.9 | μL |
| 20 ng/μL PCR product | 1.0 | μL |
| ddH₂O | 17.9 | μL |

Reaction conditions:
   1. 98° C. 10 minutes
   2 to 5. 98° C. 10 minutes, 58.4° C. 10 minutes, 72° C. 15 minutes: 19 cycles
   6. 72° C. 5 minutes
   7. 4° C. ∞.

Next, the PCR product (Illumina library) was subjected to paired-end sequencing using Illumina HiSeq (Illumina) to confirm the presence or absence of genome editing by the guide RNA. In HEK293Ta cells obtained by transfecting Array vector lib and Single vector lib, it was confirmed that cytosine (C) in the base sequence of the target region of guide RNA at 96 sites was replaced with thymine (T).

Also, based on each sequence result, the probability that cytosine (C) in the base sequence of the target region of the guide RNA at 96 sites was replaced with thymine (T) was defined as the base editing rate. FIG. 15 shows the sorted editing efficiencies of the top 26 sites with the highest base editing rates among the 96 guide RNA target regions for transfected Array vector lib, Single vector lib, and Single liner DNA lib.

As shown in FIG. 15, transfection using a vector (Array vector) in which multiple gRNAs were inserted as an array showed higher editing efficiency than transfection using a mixture of vectors containing individual gRNAs or fragments containing individual gRNAs. The method for producing a ligated DNA of the present invention (FRACTAL assembly method) makes it possible to easily prepare a vector with such a high editing efficiency and a vector that can be used for preparing such a vector.

2. Preparation of TALE Repeat Unit Array Vector (FRACTAL Assembly Method)

The sequence encoding the TALE repeat unit was divided into three fragments a, b, and c, and the method for producing linked DNA of the present invention (FRACTAL assembly method) was used to prepare a TALE repeat unit array vector in which multiple TALE repeat units were integrated into one vector. In the following examples, there is one kind for each of the fragments a, b, and c, but for example, by mixing multiple types of fragments with partially different amino acids as fragment a, it is possible to accumulate sequences encoding various TALE repeat units.

2.1 Amplification of DNA Fragments Containing TALE Repeat Unit Fragments and Selectable Marker Sequences

Plasmid pNM1088 (Spec^{R}) was used as a template, and the PCR method was used to amplify a forward primer containing recognition sequences (SacI, BsaI, BbsI, and AgeI) of restriction enzymes SacI, BsaI, BbsI, and AgeI and TALE repeat unit fragments a, b, and c (TALE_rptuinit1L (SEQ ID NO: 23, including TALE repeat unit fragment a); TALE_rptuinit2L (SEQ ID NO: 24, including TALE repeat unit fragment b); and TALE_rptuinit3L (SEQ ID NO: 25, including TALE repeat unit fragment c)) and reverse primer SpecR_CmR_common_RV (SEQ ID NO: 26) containing recognition sites (SalI, BsaI, BbsI, NheI) for restriction enzymes SalI, BsaI, BbsI, and NheI. As a result, a first DNA fragment containing “5′-SacI-BsaI-TALE repeat unit fragment (a or b or c)-BbsI-AgeI-Spec^{R}-NheI-BbsI-BsaI-SalI-3′” was obtained.

Plasmid pNM1089 (ccdB+Cm^{R}) was used as a template, and the PCR method was used to amplify a forward primer ccdBCmR_Fw containing recognition sequences (SacI, BsaI, BbsI, and AgeI) for restriction enzymes SacI, BsaI, BbsI, and AgeI (SEQ ID NO: 27) and a reverse primer containing recognition sites (SalI, BsaI, BbsI, and NheI) for restriction enzymes SalI, BsaI, BbsI, and NheI and TALE repeat unit fragments a, b, and c (TALE_rptuinit1R (SEQ ID NO: 28, including TALE repeat unit fragment a); TALE_rptuinit2R (SEQ ID NO: 29, including TALE repeat unit fragment b); and TALE_rptuinit3R (SEQ ID NO: 30, including TALE repeat unit fragment c)). As a result, a second DNA fragment containing “5′-SacI-BsaI-BbsI-AgeI-ccdB+Cm^{R}-NheI-BbsI-TALE repeat unit fragment (a or b or c)-BsaI-SalI-3′” was obtained. Note that the 3′-side of the TALE repeat unit fragment a and the 5′-side of the fragment b; and the 5′-side of the TALE repeat unit fragment b and the 3′-side of the fragment c are made into a sequence with homologous protruding ends cleaved with restriction enzyme BsaI or BbsI (FIG. 8). The PCR conditions are shown below.

[PCR Conditions]
Reaction solution (Total: 20 μL):

| | | |
|---|---|---|
| 5 × GC buffer | 4.0 | μL |
| 2.5 μM dNTPs | 0.4 | μL |
| Phusion DNA polymerase | 0.4 | μL |
| 10 μM forward primer | 1.0 | μL |
| 10 μM reverse primer | 1.0 | μL |
| 100 pg/μL plasmid | 1.0 | μL |
| ddH₂O | 12.2 | μL |

Reaction conditions:
   1. 95° C. 30 seconds
   2 to 5. 95° C. 10 seconds, 58° C. 15 seconds, 72° C. 1.5 minutes: 30 cycles
   6. 72° C. 10 minutes
   7. 4° C. ∞.

2.2 Creation of Toolkit Vectors

<Toolkit Vectors 1 and 2>

(Restriction Enzyme Treatment and Ligation)

The PCR product of 2.1 above was cleaved with restriction enzymes SacI (SacI-HF, NEB) and SalI (SalI-HF, NEB) to obtain a Donor DNA. In addition, as a host DNA, pUC19 was cleaved with restriction enzymes SacI and SalI. The first DNA fragment was ligated into pUC19 to obtain toolkit vector 1. Also, the second DNA fragment was ligated to pUC19 to obtain toolkit vector 2. The restriction enzyme treatment conditions and ligation conditions are shown below.

[Restriction Enzyme Treatment Conditions]

### [SacI/SalI]

Reaction solution (Total: 50 μL):

| | | |
|---|---|---|
| 10 × CutSmart Buffer | 5 | μL |
| SacI (20,000 units/mL) | 1 | μL |
| SalI (20,000 units/ml) | 1 | μL |
| PCR product or pUC19 | 5 | μg |

| | |
|---|---|
| ddH₂O | balance |

Reaction conditions for Donor DNA
   1. 37° C. 2 hours
   2. 1 μL CIP was added to 50 μL reaction solution
   3. 37° C. 30 minutes
Reaction conditions for Host DNA
   1. 37° C. 2 hours.

[Ligation Conditions]
Reaction solution (Total: 20 μL):
   When the selectable marker gene contained in Donor DNA is Spec^{R}

| | | |
|---|---|---|
| 10 × ligation buffer | 1 | μL |
| Donor DNA | 500 | ng |
| Host DNA | 50 | ng |
| T4 DNA Ligase (500 U/μL) | 1 | μL |

| | |
|---|---|
| ddH₂O | balance |

When the selectable marker gene contained in Donor DNA is ccdB+Cm^{R}

| | | |
|---|---|---|
| 10 × ligation buffer | 1 | μL |
| Donor DNA | 250 | ng |
| Host DNA | 50 | ng |
| T4 DNA Ligase (500 U/μL) | 0.1 | μL |

| | |
|---|---|
| ddH₂O | balance |

Reaction conditions
   1. 16° C. 1 hour
   2. 4° C. ∞.

(Transformation)

The ligation products above were transformed into E. coli, and a drug-selective medium containing antibiotics corresponding to the selectable marker genes contained in Donor DNA was used to select E. coli containing the desired toolkit vector. First, when the selectable marker gene was Spec^{R}, 1.5 μL of the ligation product was added to 20 μL NEB 5-alpha Competent E. coli (NEB). In addition, when the selectable marker gene was ccdB+Cm^{R}, 1.5 μL of the ligation reaction solution was added to 20 μL One Shot™ ccdB Survival™ 2 T1^{R} Competent Cells (Invitrogen). Then, these were allowed to stand on ice for 30 minutes, then incubated (heat shock) in a 42° C. water bath for 30 seconds, and then allowed to stand on ice for 2 minutes. Then, 250 μL of Soc medium was added to each of them, and after incubation at 37° C. for 2 hours, all of the incubated culture solutions were seeded on LB agar medium containing antibiotics corresponding to the selectable marker genes. When the selectable marker gene contained in Donor DNA was Spec^{R}, the antibiotics were ampicillin (Amp) and spectinomycin (Spec), and when the selectable marker gene contained in Donor DNA was ccdB+Cm^{R}, the antibiotics are ampicillin (Amp) and chloramphenicol (Cm). Then, after incubation at 37° C. overnight, the target toolkit vectors, that is, toolkit vector 1 and toolkit vector 2, were isolated from the E. coli whose growth was confirmed.

<Toolkit Vectors 3 and 4>

The toolkit vector 1 above was cleaved with restriction enzymes SacI (SacI-HF, NEB) and NheI (NheI-HF, NEB) to obtain Donor DNA, and the toolkit vector 2 above was cleaved with restriction enzymes SacI and NheI to remove ccdB+Cm^{R}, thereby obtaining Host DNA, and these were linked by ligation to obtain toolkit vector 3 containing “5′-BsaI-TALE repeat unit fragment (a or b or c)-BbsI-Spec^{R}-BbsI-TALE repeat unit fragment (a or b or c)-BsaI-3′” (SacI, AgeI, NheI, and SalI are not described because they are not used later).

In addition, the toolkit vector 2 above was cleaved with restriction enzymes AgeI (AgeI-HF, NEB) and SalI (SalI-HF, NEB) to obtain Donor DNA, and the toolkit vector 1 above was cleaved with restriction enzymes AgeI and SalI to remove Spec^{R}, thereby obtaining Host DNA, and these were linked by ligation to obtain toolkit vector 4 containing “5′-BsaI-TALE repeat unit fragment (a or b or c)-BbsI-ccdB+Cm^{R}-BbsI-TALE repeat unit fragment (a or b or c)-BsaI-3′” (SacI, AgeI, NheI, and SalI are not described because they are not used later).

In each toolkit vector, the combination of the TALE repeat unit fragment on the 3′-side and the TALE repeat unit fragment on the 5′-side of the selectable marker genes (Spec^{R}, ccdB+Cm^{R}) after ligation was made to be a-c, b-b, or c-a (first stage ligation). The restriction enzyme treatment conditions are shown below. The reaction conditions for restriction enzyme treatment and ligation conditions are as shown in

### <Toolkit Vectors 1 and 2>.

[Restriction Enzyme Treatment Conditions]
Reaction solution (Total: 50 μL):

[SacI/NheI]

| | | |
|---|---|---|
| 10 × CutSmart Buffer | 5 | μL |
| SacI (20,000 units /mL) | 1 | μL |
| NheI (20,000 units/ml) | 1 | μL |
| toolkit vectors 1 and 2 | 5 | μg each |

| | |
|---|---|
| ddH₂O | balance |

[AgeI/SalI]

| | | |
|---|---|---|
| 10 × CutSmart Buffer | 5 | μL |
| AgeI (20,000 units/mL) | 1 | μL |
| SalI (20,000 units/ml) | 1 | μL |
| toolkit vectors 1 and 2 | 5 | μg each |

| | |
|---|---|
| ddH₂O | balance |

Also, the ligation products above were transformed into E. coli, and a drug-selective medium containing antibiotics corresponding to the selectable marker genes contained in Donor DNA was used to select E. coli containing the desired toolkit vector. The transformation conditions are the same as <Toolkit Vectors 1 and 2>.

2.3 Toolkit Vector Cleaving Process

As a donor vector containing the TALE repeat unit fragments to be added, toolkit vector 4 was cleaved with restriction enzyme BsaI (BsaI-HFv2, NEB), and as a host vector for receiving the TALE repeat unit fragments, toolkit vector 3 was cleaved with restriction enzyme BbsI (BbsI-HF, NEB). The restriction enzyme treatment conditions are shown below.

[Restriction Enzyme Treatment Conditions]

### [BsaI for Donor DNA]

Reaction solution (Total: 50 μL):

| | | |
|---|---|---|
| 10 × CutSmart Buffer | 5 | μL |
| BsaI (20,000 units/ml) | 1 | μL |
| donor vector | 5 | μg |

| | |
|---|---|
| ddH₂O | balance |

Reaction conditions
   1. 37° C. 2 hours
   1 μL, CIP was added to 50 μL reaction solution
   3. 37° C. 30 minutes

### [BbsI for Host DNA]

Reaction solution (Total: 50 μL):

| | | |
|---|---|---|
| 10 × CutSmart Buffer | 5 | μL |
| BbsI (20,000 units/ml) | 1 | μL |
| host vector | 5 | μg |

| | |
|---|---|
| ddH₂O | balance |

Reaction conditions
   1. 37° C. 2 hours.

### 2.4 Linking TALE Repeat Unit Fragments by Ligation

A fragment having two TALE repeat unit fragments (“5′-TALE repeat unit fragment (a or b or c)-BbsI-ccdB+Cm^{R}-BbsI-TALE repeat unit fragment (a or b or c)-3′”) and a fragment obtained by removing the selectable marker gene (Spec^{R}) from the host vector (“TALE repeat unit fragment (a or b or c)-3′/5′-TALE repeat unit fragment (a or b or c)”), which were excised from the donor vector in 2.3 above, were recovered, and were ligated so that the combination of the 3′-side TALE repeat unit fragment and the 5′-side TALE repeat unit fragment of ccdB+Cm^{R} after ligation would be ab-bc, thereby obtaining the target vector (second stage ligation). The ligation conditions are as shown in 2.2 above. The ligation product was transformed into E. coli, and E. coli containing the target product was selected using an appropriate drug selection medium to obtain the target vector. The transformation method is the same as 2.2.

2.5 Repetition of Linking TALE Repeat Unit Fragments by Ligation

The above 2.2 to 2.4 were repeated for ligation so that the combination of the 3′-side TALE repeat unit fragment and the 5′-side TALE repeat unit fragment of ccdB+Cm^{R} after ligation would be abc-abc, thereby obtaining the target vector (third stage ligation). The above was further repeated for ligation so that The 3′-side TALE repeat unit fragment abc and the 5′-side TALE repeat unit fragment abc of ccdB+Cm^{R} after ligation would be linked in order, thereby obtaining vector in which a total of 48 TALE repeat unit fragments were ligated to the 5′-side and 3′-side of ccdB+Cm^{R}, 24 fragments each (8 repeats of abc).

2.6 Sequence Confirmation by Sanger Sequencing Method

As for the vector obtained in 2.5 above in which a total of 48 TALE repeat unit fragments were ligated to the 5′-side and 3′-side of ccdB+Cm^{R}, 24 fragments each (8 repeats of abc), the sequences of 24 fragments on the 5′-side (8 repeats of abc) and 24 fragments of the 3′-side (8 repeats of abc) of ccdB+Cm^{R} were subjected to the Sanger sequencing method, and it was confirmed that a vector was indeed obtained to which the target TALE repeat unit fragment was ligated (vector containing “5′-BsaI-TALE repeat unit fragment×24 (abc-abc-abc-abc-abc-abc-abc-abc)-BbsI-ccdB+Cm^{R}-BbsI-TALE repeat unit fragment×24 (abc-abc-abc-abc-abc-abc-abc-abc)-BsaI-3′”).

2.7 Removal of ccdB+Cm^{R}

The vector whose sequence was confirmed in 2.6 above was cleaved with the restriction enzyme BbsI and subjected to self-ligation to remove ccdB+Cm^{R}, thereby obtaining a TALE repeat unit array vector in which 48 TALE repeat unit fragments were linked together (16 consecutive abc links). The restriction enzyme treatment conditions are as shown in 2.3 above. The method for producing a ligated DNA of the present invention (FRACTAL assembly method) also makes it possible to prepare a TALE repeat unit array in which TALE repeat units are continuously ligated in this way.

### INDUSTRIAL APPLICABILITY

As described above, according to the present invention, it is possible to provide a method for producing a ligated DNA capable of accurately and efficiently ligating several tens or more of DNA fragments easily, and vector combinations for use therein.

In addition, according to the present invention, even fragments in which the same sequence such as a repeat sequence appears many times can be continuously ligated and can be used as they are for another assembly, so that reusability is high as well. Furthermore, since the probability is low of generating a non-targeted product due to non-specific ligation or the like, it is also possible to reduce the labor and time required for quality inspection. According to the present invention, it is also possible to prepare a vector library for efficient multiple genome editing and a pooled library for isolating a desired clone by the PCR method.

### Sequence Listing Free Text

SEQ ID NO: 1
<223> primer “DG012”
SEQ ID NO: 2
<223>primer “DG011”
SEQ ID NO: 3
<223>primer “DG009”
SEQ ID NO: 4
<223>primer “DG010”
SEQ ID NO: 5
<223>primer “DG007”
SEQ ID NO: 6
<223>primer “DG008”
SEQ ID NO: 7
<223>primer “DG001”
SEQ ID NO: 8
<223>primer “DG002”
SEQ ID NO: 9
<223>primer “DG003”
SEQ ID NO: 10
<223>primer “DG004”
SEQ ID NO: 11
<223>primer “DG013”
SEQ ID NO: 12
<223>primer “DG015”
SEQ ID NO: 13
<223>primer “DG021”
SEQ ID NO: 14
<223>primer “M13-Fw”
SEQ ID NO: 15
<223>primer “DG020”
SEQ ID NO: 16
<223>primer “DG006”
SEQ ID NO: 17
<223>primer “NM_ABC001Fw”
SEQ ID NO: 18
<223>primer “NM_ABC001Rv”
SEQ ID NO: 19
<223>primer “NM_ABC_gt_1_Fw”
SEQ ID NO: 20
<223>primer “NM_ABC_gt_1_Rv”
SEQ ID NO: 21
<223>primer “BC_0074”
<223> n is a, c, g, or t
SEQ ID NO: 22
<223>primer “BC_0075”
<223> n is a, c, g, or t
SEQ ID NO: 23
<223>primer “TALE_rptuinit1L”
SEQ ID NO: 24
<223>primer “TALE_rptuinit2L”
SEQ ID NO: 25
<223>primer “TALE_rptuinit3L”
SEQ ID NO: 26
<223>primer “SpecR_CmR_common_RV”
SEQ ID NO: 27
<223>primer “ccdBCmR_Fw”
SEQ ID NO: 28
<223>primer “TALE_rptuinit1R”
SEQ ID NO: 29
<223>primer “TALE_rptuinit2R”
SEQ ID NO: 30
<223>primer “TALE_rptuinit3R”

1. A method for producing a ligated DNA formed by ligating DNA fragments, comprising:
(a1) a step a1 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(1)₁) a step b1 of treating the first vector with the first restriction enzyme and the second restriction enzyme to obtain a first vector fragment composed of the structure: 5′-D(i)-R2-M1-R2′-D(ii)-3′;
(c1) a step c1 of treating the second vector with the third restriction enzyme and the fourth restriction enzyme to obtain a second vector fragment with the removed structure: 5′-R2-M2-R2′-3′; and
(d1) a step d1 of ligating the first vector fragment obtained in step b1 and the second vector fragment obtained in step c1 by a ligation reaction to generate a third vector containing the following structure (3):
5′-R1-D(i)₁-R2-M1-R2′-D(ii)₁-R1′-3′ (3)
wherein D(i)₁ represents a DNA fragment containing the following structure: 5′-D(iii)-D(i)-3′, and D(ii)i represents a DNA fragment containing the following structure: 5′-D(ii)-D(iv)-3′.

2. The method for producing a ligated DNA according to claim 1, further comprising: after step d1, a step of transforming a ligation reaction product into a host; and a step of using expression of the first selectable marker gene as an index to select a host introduced with the third vector.

3. The method for producing a ligated DNA according to claim 1, further comprising: after step d1, a step of treating the third vector with the third restriction enzyme and the fourth restriction enzyme to remove the structure: 5′-R2-M1-R2′-3′, thereby generating a fifth vector containing the structure: 5′-R1-D(i)₁-D(ii)₁-R1′-3′.

4. The method for producing a ligated DNA according to claim 1, further comprising: using the third vector generated in step d1 as the first vector in step a1 and repeating steps a1 to d1 for an additional n cycles (1+n cycles in total) to generate a third′ vector containing the structure (3′):
5′-R1-D(i)₁₊ₙ-R2-M1-R2′-D(ii)₁₊ₙ-R1′-3′ (3′)
wherein D(i)₁₊ₙ represents a DNA fragment containing the structure obtained at cycle 1+n: 5′-D(iii)-D(i)ₙ-3; D(ii)₁₊ₙ represents a DNA fragment containing the structure obtained at cycle 1+n: 5′-D(ii)ₙ-D(iv)-3; n represents a natural number; between the cycles, D(iii) of the second vector may be the same or different from each other; and between the cycles, D(iv) of the second vector may be the same or different from each other.

5. A method for producing a ligated DNA formed by ligating DNA fragments, comprising:
(a2) a step a2 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(b2) a step b2 of treating the second vector with the first restriction enzyme and the second restriction enzyme to obtain a second vector fragment composed of the structure: 5′-D(iii)-R2-M2-R2′-D(iv)-3;
(c2) a step c2 of treating the first vector with the third restriction enzyme and the fourth restriction enzyme to obtain a first vector fragment with the removed structure: 5′-R2-M1-R2′-3; and
(d2) a step d2 of ligating the second vector fragment obtained in step b2 and the first vector fragment obtained in step c2 by a ligation reaction to generate a fourth vector containing the following structure (4):
5′-R1-D(iii)₁-R2-M2-R2′-D(iv)₁-R1′-3′ (4)
wherein D(iii)₁ represents a DNA fragment containing the following structure: 5′-D(i)-D(iii)-3′, and D(iv)₁ represents a DNA fragment containing the following structure: 5′-D(iv)-D(ii)-3′.

6. The method for producing a ligated DNA according to claim 5, further comprising: after step d2, a step of transforming a ligation reaction product into a host; and a step of using expression of the second selectable marker gene as an index to select a host introduced with the fourth vector.

7. The method for producing a ligated DNA according to claim 5, further comprising: after step d2, a step of treating the fourth vector with the third restriction enzyme and the fourth restriction enzyme to remove the structure: 5′-R2-M2-R2′-3′, thereby generating a sixth vector containing the structure: 5′-R1-D(iii)₁-D(iv)₁-R1′-3′.

8. The method for producing a ligated DNA according to claim 5, further comprising: using the fourth vector generated in step d2 as the second vector in step a2 and repeating steps a2 to d2 for an additional n cycles (1+n cycles in total) to generate a fourth′ vector containing the structure (4′):
5′-R1-D(iii)₁₊ₙ-R2-M2-R2′-D(iv)₁₊ₙ-R1′-3′ (4′)
wherein D(iii)₁₊ₙ represents a DNA fragment containing the structure obtained at cycle 1+n: 5′-D(i)-D(iii)ₙ-3′; D(iv)₁₊ₙ, represents a DNA fragment containing the structure obtained at cycle 1+n: 5′-D(iv)ₙ-D(ii)-3′; n represents a natural number; between the cycles, D(i) of the first vector may be the same or different from each other; and between the cycles, D(ii) of the first vector may be the same or different from each other.

9. The method for producing a ligated DNA according to claim 1, wherein the second vector in step a1 is a fourth vector containing the following structure (4):
5′-R1-D(iii)₁-R2-M2-R2′-D(iv)₁-R1′-3′ (4)
wherein D(iii)₁ represents a DNA fragment containing the following structure: 5′-D(i)-D(iii)-3′, and D(iv)₁ represents a DNA fragment containing the following structure: 5′-D(iv)-D(ii)-3′, formed by a process comprising:
(a2) a step a2 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(b2) a step b2 of treating the second vector with the first restriction enzyme and the second restriction enzyme to obtain a second vector fragment composed of the structure: 5′-D(iii)-R2-M2-R2′-D(iv)-3′;
(c2) a step c2 of treating the first vector with the third restriction enzyme and the fourth restriction enzyme to obtain a first vector fragment with the removed structure: 5′-R2-M1-R2′-3′; and
(d2) a step d2 of ligating the second vector fragment obtained in step b2 and the first vector fragment obtained in step c2 by a ligation reaction to generate the fourth vector.

10. The method for producing a ligated DNA according to claim 5, wherein the first vector in step a2 is a third vector containing the following structure (3):
5′-R1-D(i)₁-R2-M1-R2′-D(ii)₁-R1′-3′ (3)
wherein D(i)₁ represents a DNA fragment containing the following structure: 5′-D(iii)-D(i)-3′, and D(ii)₁ represents a DNA fragment containing the following structure: 5′-D(ii)-D(iv)-3′, prepared by a process comprising:
(a1) a step a1 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein, R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(b1) a step b1 of treating the first vector with the first restriction enzyme and the second restriction enzyme to obtain a first vector fragment composed of the structure: 5′-D(i)-R2-M1-R2′-D(ii)-3′;
(c1) a step c1 of treating the second vector with the third restriction enzyme and the fourth restriction enzyme to obtain a second vector fragment with the removed structure: 5′-R2-M2-R2′-3′, and
(d1) a step d1 of ligating the first vector fragment obtained in step b1 and the second vector fragment obtained in step c1 by a ligation reaction to generate the third vector.

11. The method for producing a ligated DNA according to claim 1, wherein
the first restriction enzyme is a type IIS restriction enzyme that cleaves the 3′-side of R1, and the second restriction enzyme is a type IIS restriction enzyme that cleaves the 5′-side of R1′, and/or
the third restriction enzyme is a type IIS restriction enzyme that cleaves the 5′-side of R2, and the fourth restriction enzyme is a type IIS restriction enzyme that cleaves the 3′-side of R2′.

12. The method for producing a ligated DNA according to claim 1, wherein
a third selectable marker gene, which is a selectable marker gene with an opposite action to that of the first selectable marker gene, is further inserted between R2 and R2′ of the first vector, and/or
a fourth selectable marker gene, which is a selectable marker gene with an opposite action to that of the second selectable marker gene and can be the same as or different from the third selectable marker gene, is further inserted between R2 and R2′ of the second vector.

13. The method for producing a ligated DNA according to claim 1, wherein
a recognition sequence of a fifth restriction enzyme different from R1, R1′, R2, and R2′ is further set at a site other than the structure (1) in the first vector, and
a recognition sequence of a sixth restriction enzyme different from R1, R1′, R2, R2′, and the recognition sequence of the restriction enzyme is further set at a site other than the structure (2) in the second vector.

14-15. (canceled)

16. The method for producing a ligated DNA according to claim 1, wherein the second vector in step a1 is a fourth′ vector containing the structure (4′):
5′-R1-D(iii)₁₊ₙ-R2-M2-R2′-D(iv)₁₊ₙ-R1′-3′ (4′)
wherein D(iii)₁₊ₙ represents a DNA fragment containing the structure obtained at cycle 1+n: 5′-D(i)-D(iii)ₙ-3; D(iv)₁₊ₙ, represents a DNA fragment containing the structure obtained at cycle 1+n: 5′-D(iv)ₙ-D(ii)-3′; n represents a natural number; between the cycles, D(i) of the first vector may be the same or different from each other; and between the cycles, D(ii) of the first vector may be the same or different from each other, prepared by a process comprising:
(a2) a step a2 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(b2) a step b2 of treating the second vector with the first restriction enzyme and the second restriction enzyme to obtain a second vector fragment composed of the structure: 5′-D(iii)-R2-M2-R2′-D(iv)-3;
(c2) a step c2 of treating the first vector with the third restriction enzyme and the fourth restriction enzyme to obtain a first vector fragment with the removed structure: 5′-R2-M1-R2′-3; and
(d2) a step d2 of ligating the second vector fragment obtained in step b2 and the first vector fragment obtained in step c2 by a ligation reaction to generate a fourth vector containing the following structure (4):
5′-R1-D(iii)₁-R2-M2-R2′-D(iv)₁-R1′-3′ (4)
wherein D(iii)₁ represents a DNA fragment containing the following structure: 5′-D(i)-D(iii)-3′, and D(iv)₁ represents a DNA fragment containing the following structure: 5′-D(iv)-D(ii)-3;
a step using the fourth vector generated in step d2 as the second vector in step a2 and repeating steps a2 to d2 for an additional n cycles (1+n cycles in total) to generate the fourth′ vector.

17. The method for producing a ligated DNA according to claim 5, wherein the first vector in step a2 is a third′ vector containing the structure (3′):
5′-R1-D(i)₁₊ₙ-R2-M1-R2′-D(ii)₁₊ₙ-R1′-3′ (3′)
wherein D(i)₁₊ₙ represents a DNA fragment containing the structure obtained at cycle 1+n: 5′-D(iii)-D(i)ₙ-3; D(ii)₁₊ₙ represents a DNA fragment containing the structure obtained at cycle 1+n: 5′-D(ii)ₙ-D(iv)-3′; n represents a natural number; between the cycles, D(iii) of the second vector may be the same or different from each other; and between the cycles, D(iv) of the second vector may be the same or different from each other, prepared by a process comprising:
(a1) a step a1 of preparing a first vector containing the following structure (1) and a second vector containing the following structure (2):
5′-R1-D(i)-R2-M1-R2′-D(ii)-R1′-3′ (1)
5′-R1-D(iii)-R2-M2-R2′-D(iv)-R1′-3′ (2)
wherein R1 represents a recognition sequence of a first restriction enzyme; R1′ represents a recognition sequence of a second restriction enzyme; R2 represents a recognition sequence of a third restriction enzyme different from the first restriction enzyme and the second restriction enzyme; R2′ represents a recognition sequence of a fourth restriction enzyme different from the first restriction enzyme and the second restriction enzyme; M1 represents a first selectable marker gene; M2 represents a second selectable marker gene different from the first selectable marker gene; D(i) to D(iv) each independently represent a DNA fragment for ligation; D(i) and D(ii) may be either one, and D(iii) and D(iv) may be either one; the first restriction enzyme cleaves inside of R1 or a 3′-side of R1, and the second restriction enzyme cleaves inside of R1′ or a 5′-side of R1′, and the first restriction enzyme and the second restriction enzyme may be the same or different; the third restriction enzyme cleaves inside of R2 or a 5′-side of R2, and the fourth restriction enzyme cleaves inside of R2′ or a 3′-side of R2′, and the third restriction enzyme and the fourth restriction enzyme may be the same or different;
(b1) a step b1 of treating the first vector with the first restriction enzyme and the second restriction enzyme to obtain a first vector fragment composed of the structure: 5′-D(i)-R2-M1-R2′-D(ii)-3′;
(c1) a step c1 of treating the second vector with the third restriction enzyme and the fourth restriction enzyme to obtain a second vector fragment with the removed structure: 5′-R2-M2-R2′-3′; and
(d1) a step d1 of ligating the first vector fragment obtained in step b1 and the second vector fragment obtained in step c1 by a ligation reaction to generate a third vector containing the following structure (3):
5′-R1-D(i)₁-R2-M1-R2′-D(ii)₁-R1′-3′ (3)
wherein D(i)₁ represents a DNA fragment containing the following structure: 5′-D(iii)-D(i)-3′, and D(ii)₁ represents a DNA fragment containing the following structure: 5′-D(ii)-D(iv)-3;
a step using the third vector generated in step d1 as the first vector in step a1 and repeating steps a1 to d1 for an additional n cycles (1+n cycles in total) to generate the third′ vector.

18. The method for producing a ligated DNA according to claim 5, wherein
the first restriction enzyme is a type IIS restriction enzyme that cleaves the 3′-side of R1, and the second restriction enzyme is a type IIS restriction enzyme that cleaves the 5′-side of R1′, and/or
the third restriction enzyme is a type IIS restriction enzyme that cleaves the 5′-side of R2, and the fourth restriction enzyme is a type IIS restriction enzyme that cleaves the 3′-side of R2′.

19. The method for producing a ligated DNA according to claim 5, wherein
a third selectable marker gene, which is a selectable marker gene with an opposite action to that of the first selectable marker gene, is further inserted between R2 and R2′ of the first vector, and/or
a fourth selectable marker gene, which is a selectable marker gene with an opposite action to that of the second selectable marker gene and can be the same as or different from the third selectable marker gene, is further inserted between R2 and R2′ of the second vector.

20. The method for producing a ligated DNA according to claim 5, wherein
a recognition sequence of a fifth restriction enzyme different from R1, R1′, R2, and R2′ is further set at a site other than the structure (1) in the first vector, and
a recognition sequence of a sixth restriction enzyme different from R1, R1′, R2, R2′, and the recognition sequence of the restriction enzyme is further set at a site other than the structure (2) in the second vector.
